(19) United States
(12) Patent Application Publication
(10) Pub. No.: US 2006/0122387 A1
(43) Pub. Date: Jun. 8, 2006

(54) Derivatives of chromen-2-one as inhibitors of vegf production in mammalian cells

(75) Inventors: Ernesto Menta, Cernusco Sul Naviglio (IT); Giovanni Da Re, Milan (IT); Mario Grugni, Milan (IT)

Correspondence Address: DONALD W. WYATT;CELL THERAPEUTICS, INC., 501 ELLIOTT AVENUE WEST, #400, SEATTLE, WA, 98119, US

(73) Assignee: CTI Europe S.r.l.

(21) Appl. No.: 10/517,805

(22) PCT Filed: Jun. 12, 2003
(86) PCT No.: PCT/EP03/06191

§ 371 Date: Dec. 5, 2005

Related U.S. Application Data
(60) Provisional application No. 60/387,917, filed on Jun. 13, 2002.

(51) Int. Cl. C07D 417/02; C07D 413/02; C07D 405/02

(52) U.S. Cl. 544/60; 544/151; 544/375; 549/283

(57) ABSTRACT

The compounds of formula (I) wherein A and R1-R5 are as defined in the description, are inhibitors of Vascular Endothelial Growth Factor and are useful as angiogenesis inhibitors and antiproliferative agents.

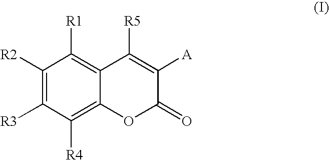

### TECHNICAL FIELD

This invention pertains generally to the field of antiproliferative compounds, and more specifically to certain active compounds which inhibit Vascular Endothelial Growth Factor (VEGF) production and thereby inhibit angiogenesis, tumorigenesis, and proliferative conditions, such as cancer. The present invention also pertains to pharmaceutical compositions comprising such compounds, and the use of such compounds and compositions, both in vitro and in vivo, to inhibit VEGF production, and to inhibit angiogenesis, tumorigenesis, and proliferative conditions, such as cancer.

### BACKGROUND OF THE INVENTION

Mammalian cells require oxygen and nutrients in order to survive, and are therefore always located within 100-200 μm from the nearest blood vessel. This is equally true for tumour cells—in order to grow beyond about 1 mm³, they require growth of new blood vessels. This vascularization process, called angiogenesis, is a hallmark of all solid tumours, and has become a rich area of research, due to the potential for therapeutic intervention. As well as cancer, angiogenesis plays a role in diabetic retinopathy, rheumatoid arthritis, psoriasis, atherosclerosis and restenosis (reviewed by Folkman).

A large number of putative pro-angiogenic factors have been characterised (reviewed by Augustin); however, the most relevant for tumour angiogenesis are the peptides belonging to the vascular endothelial growth factor (VEGF) family. There are at least four VEGFs known, termed A to D. VEGFs A and B seem to be the main players in haemangiogenesis, whereas the likely role of VEGFs C and D may be in lymphangiogenesis.

VEGFs are secreted by tumour cells in response to diverse stimuli, including hypoxia, acidic pH conditions, and activation of proto-oncogenes such as c-Src. The molecular targets of VEGFs are specific receptors, found on the surface of vascular endothelial cells. There are at least 3 VEGF receptors, called VEGF-R1(flt-1), VEGF-R2(flk1/KDR) and VEGF-R3 (flt-4).

Genbank accession nos: human VEGF-R1 P17948

human VEGF-R2 P35968

human VEGF-R3 P35916

human VEGF-A AAA35789

human VEGF-B XP_{—}006539

human VEGF-C XP_{—}003456

human VEGF-D NP_{—}004460

The isocoumarin derivative 8-hydroxy-6-methoxy-,alpha, -methyl-1-oxo-1H-2-benzopyran-3-acetic acid, known as NM-3, is reported to be an inhibitor of VEGF secretion from a number of cell types, and has shown anti-angiogenic and anti tumour activity in animal models of cancer. This compound is covered by U.S. Pat. No. 6,020,363. (16). Hashimoto et al describe coumarins for the inhibition of 12-lipoxygenase, an enzyme involved in prostaglandin synthesis. These coumarins are substituted in the 3-position with optionally substituted thienyl, furyl or phenyl groups, and have cited therapeutic use in arteriosclerosis and metastasis of cancer. Further patent applications covering the use of coumarin derivatives as anti-cancer agents include those from Mladen et al (19), and Yuzo (20).

Because of the central role of VEGF in angiogenesis, it is expected that inhibitors of VEGF secretion would be beneficial in the treatment of all diseases in which angiogenesis is known to play a role. Such indications include:

Cancer

Rheumatoid arthritis (1)

Osteoarthritis (1)

Granuloma (2)

Retinal neovascularization (3)

Choroidal neovascularization (3)

Diabetic nephropathy (4)

Melorheostosis (5)

Asthma (6)

Pulmonary fibrosis (6)

Inflammation (6)

Synovitis (7)

Abortifacients (8)

Wound healing (9)

Psoriasis (10)

Endometriosis (11)

Severe ovarian hyperstimulation syndrome (11)

Myelodysplastic syndromes (12)

Haemorrhagic telengectasia (13)

Atherosclerosis (14)

Restenosis (14)

Thrombosis (14)

Crohn's disease (15)

Inflammatory bowel disease (15)

Ulcerative colitis (15)

Macular degeneration (17)

### REFERENCES

(1) Giatromanolaki, A. et al (2001) The angiogenic pathway ‘vascular endothelial growth factor/flk-1(KDR)-receptor’ in rheumatoid arthritis and osteoarthritis. J. Pathol. 194 101-108.
(2) Kimura, I. et al (2001) New triterpenes, myrrhanol A and myrrhanone A, from guggul-gum resins, and their potent anti-inflammatory effect on adjuvant-induced air-pouch granuloma of mice. Bioorg. Med. Chem. Lett. 11 985-989.
(3) Meneses, P. I. et al (2001) Recombinant angiostatin prevents retinal neovascularization in a murine proliferative retinopathy model. Gene Ther. 8 646-648.
(4) Vriese, A. S. et al (2001) Antibodies against Vascular Endothelial Growth Factor improve early renal dysfunction in experimental diabetes. J. Am. Soc. Nephrol. 12 993-1000.
(5) Hoshi, K. et al (2001) Histopathological characterization of melorheostosis. Orthopedics 24 273-277.
(6) Walsh, D. A. and Pearson, C. I. (2001) Angiogenesis in the pathogenesis of inflammatory joint and lung diseases. Arthritis Res. 3 147-153.
(7) Szekanecz, Z. and Koch, A. E. (2001) Update on synovitis. Curr. Rheumatol. Rep. 3 53-63.
(8) Sherer, D. M. and Abulafia, O. (2001) Angiogenesis during implantation, and placental and early embryonic development. Placenta 22 1-13.
(9) Lingen, M. W. (2001) Role of leukocytes and endothelial cells in the development of angiogenesis in inflammation and wound healing. Arch. Pathol. Lab. Med. 125 67-71.
(10) Nickoloff, B. J. (2000) Characterization of lymphocyte-dependent angiogenesis using a SCID mouse: human skin model of psoriasis. J. Investig. Dermatol. Symp. Proc. 5 67-73.
(11) Sherer, D. M. (2000) The role of angiogenesis in the accumulation of peritoneal fluid in benign conditions and the development of malignant ascites in the female. Gynecol. Obstet. Invest. 50 217-224.
(12) Cheson, B. D. et al (2000) Novel therapeutic agents for the treatment of myelodysplastic syndromes. Semin. Oncol. 27 560-577.
(13) Azuma, H. (2000) Genetic and molecular pathogenesis of hereditary hemorrhagic telangectasia. J. Med. Invest. 47 81-90.
(14) Eisenstein, R. (1991) Angiogenesis in arteries: review. Pharmacol. Ther. 49 1-19.
(15) Kanazawa, S. (2001) VEGF, basic-FGF and TGF-beta in Crohn's disease and ulcerative colitis: a novel mechanism of chronic intestinal inflammation. Am. J. Gastroenterol. 96 822-828.
(16) Hirano, S.-i., et al (2000) Isocoumarin derivatives and use thereof in drugs. U.S. Pat. No. 6,020,363. Feb. 1, 2000.
(17) Lip P. L. et al (2001) Age related macular degeneration is associated with increased vascular endothelial growth factor, hemorheology and endothelial dysfunction. Ophthalmology 108 705-710.
(18) Hashimoto et al (1996) Coumarin derivatives and use thereof. U.S. Pat. No. 5,574,062. Nov. 12th, 1996.
(19) Mladen et al (1997) New coumarin quinolone carboxylic acid and production thereof. JP patent application 09198505. Apr. 7th 1998.
(20) Yuzo, G. (1993). Carcinostatic agent. JP patent application 03175810. 8th Jan. 1993.

### PRIOR ART

5-coumaryl thiazoles

A series of 2-substituted 5-coumaryl thiazoles has been described (Desai et al., 1997) as fluorescent dyes:

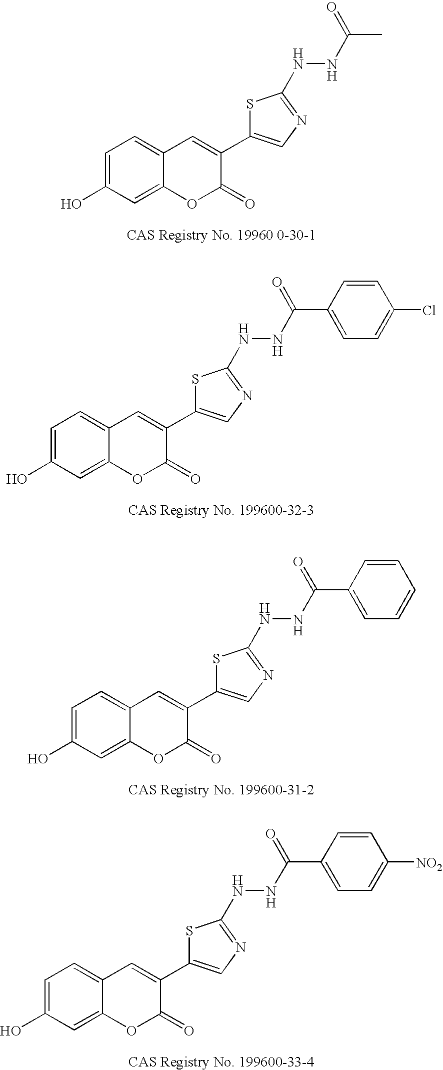

Another 2-substituted 5-coumaryl thiazole has been described (El-Morsy et al, 1988), as having bactericidal activity.

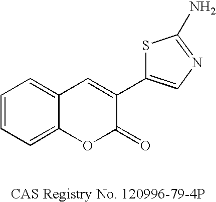

The synthesis of series of 4-methyl 5-coumaryl thiazoles has been described (Westphal, 1969):

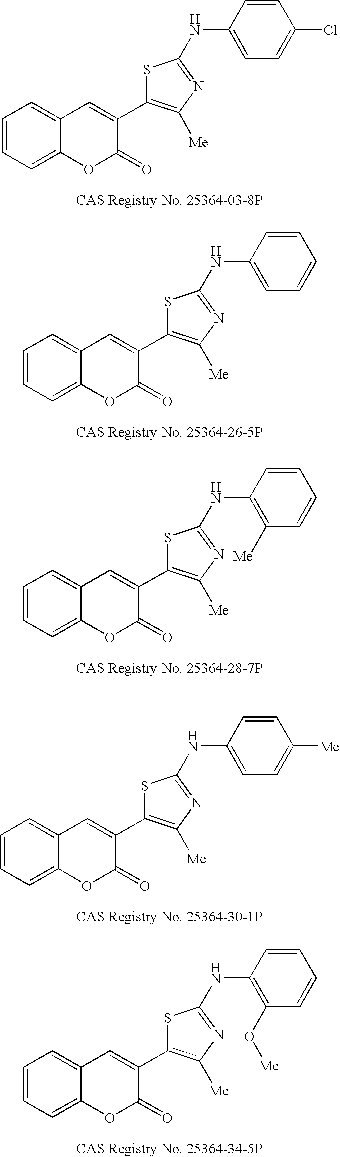

2-coumaryl thiazoles

Many 2-coumaryl thiazoles are known, almost exclusively described as dyestuffs. A small number of such compounds have been described as of in vivo utility.

Lerchen et al describe the attachment of a thiazolylcoumarin to sugar moieties to assay the tissue distribution of such glycoconjugates:

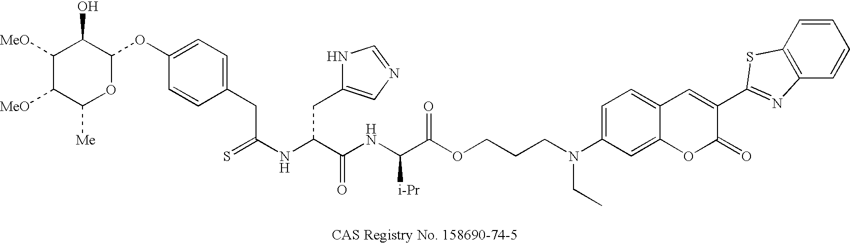

A coumaryl thiazole has been claimed as an antihelminthic (Brown, 1967):

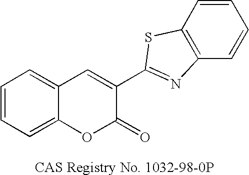

A series of 5-substituted analogues has been described as inhibitors of Macrophage Migration Inhibitory Factor (Orita et al., 2001):

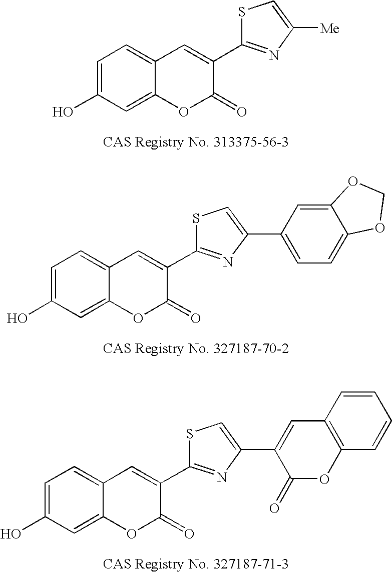

A coumarylthiazole conjugated to a saccharin moiety has been claimed as an inhibitor of the proteases chymotrypsin and elastase for the treatment of degenerative disease (Hlasta et al 1993):

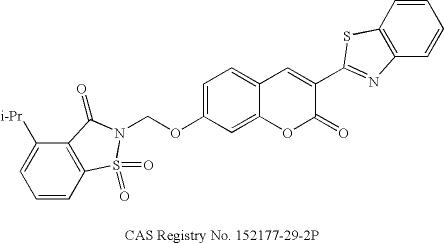

Finally, a coumarin with the relevant chemical features has been described as a bactericide (Gohar, 1990):

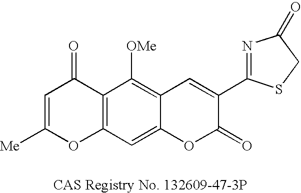

4-coumaryl thiazoles

As is the case for 2-coumarylthiazoles, there are many 4-coumarylthiazoles in the prior art. However the majority of these are for use as dyestuffs. Nevertheless there are a number of such compounds with imputed biological utility, as follows:

A series of coumarylthiazoles have been described as having bactericidal activity (Kalluraya et al, 2000)

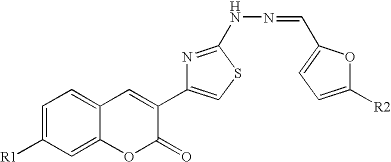

CAS Registry Nos.
52726-04-2P
365216-13-3P
365216-14-4P
365216-17-7P
365216-19-9P
365216-20-2P

4-coumarylthiazoles have also been described as inhibiting the tautomerase activity of Macrophage Migration Inhibitory Factor

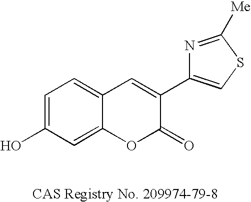

Coumarinyl thiazolinones have been described (Gursoy, 2000) as potential tuberculostatic agents:

CAS Registry Nos.

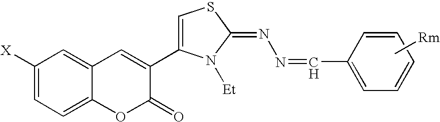

144888-10-8
268211-16-1P
268211-17-2P
268211-26-3P
268211-27-4P
268211-28-5P

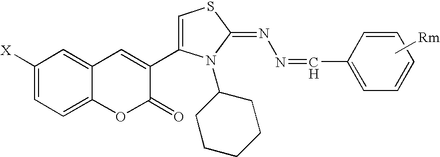

144888-11-9
144888-13-1
268211-24-1P
268211-25-2P
268211-36-5P
268211-39-8P

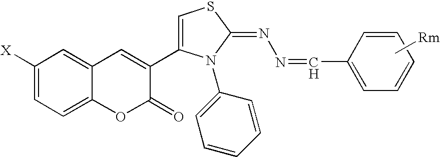

144888-15-3

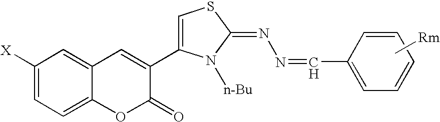

268211-21-8P
268211-22-9P
268211-23-0P
268211-24-1P
268211-32-1P
268211-33-2P
268211-34-3P

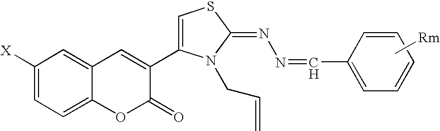

268211-18-3P
268211-19-4P
268211-20-7P
268211-29-6P
268211-30-9P
268211-31-0P

A series of triheterocyclic compounds has been synthesised as potential bactericidal agents (Kalluraya et al, 1999):

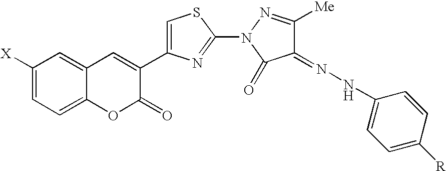

CAS Registry No.
229486-59-3P
229486-60-6P
229486-63-9P
229486-64-0P

Bactericidal activity has also been found in the following coumaryl thiazoles (Kalluraya et al., 1995):

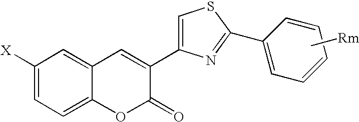

CAS Registry Nos.
88735-49-3P
88735-52-8P
175654-84-9P
175654-85-0P
175654-86-1P
175654-87-2P
175654-88-3P
175654-89-4P
175654-92-9P
175654-93-0P

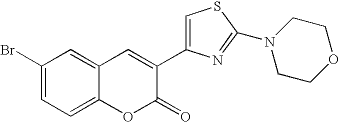

CAS Registry No.
175654-90-7P

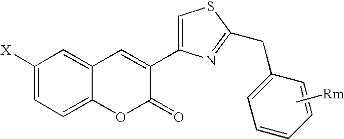

CAS Registry Nos.
175654-91-8P
175654-94-1P
175654-95-2P

Similar compounds have been described as anti-inflammatory agents (Hanmantgad et al, 1984):

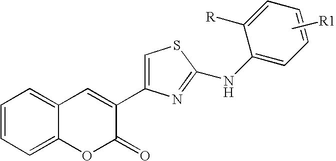

CAS Registry Nos.
97268-08-1P
97268-09-2P
97268-10-5P
97268-11-6P
97268-12-7P
97268-13-8P

Anti-inflammatory activity and potential use against allergy, anaphylaxis and arthritis has been claimed for the following coumarins (Chiarino et al, 1988)

CAS Registry Nos.

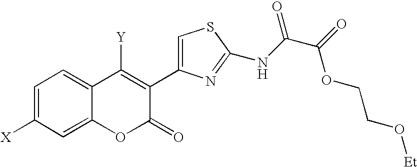

119385-94-3P
119385-93-2P
119385-96-5P

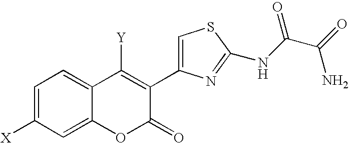

119386-00-4P
119386-01-5P
119386-02-6P

A coumaryl thiazole has been described (Yagodinets et al, 1995) as a potential microbicide:

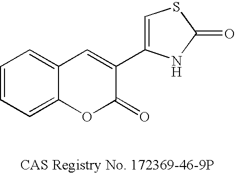

A coumarinylthiazole has been claimed as an inhibitor of bone resorption (Orme et al, 1998; Petrie et al, 1997):

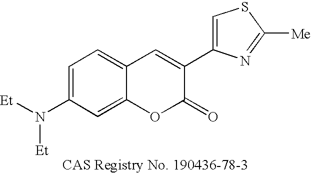

A series of triheterocyclic thiazoles has been described as potential anti-inflammatory agents (Kulkarni et al 1990).

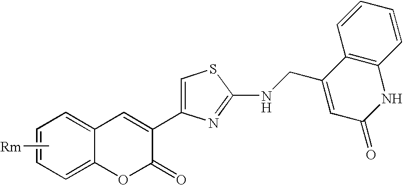

CAS Registry Nos.
132973-44-5P
132973-45-6P
132973-46-7P
132973-47-8P
132973-48-9P

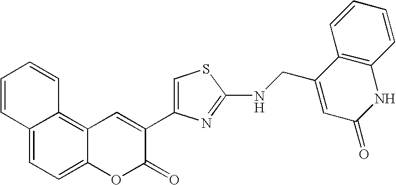

CAS Registry No.
132973-49-0P

Further coumaryl thiazoles have been investigated for potential antimicrobial activity (Hishmat et al., 1989):

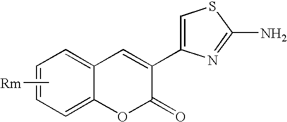

CAS Registry Nos.
126357-21-9P
126357-22-0P
126357-23-1P

Yet another antibacterial coumarin has been described (Kreutzberger et al, 1976):

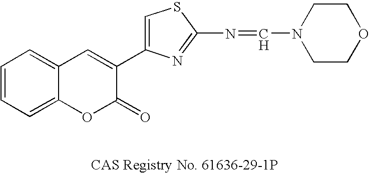

Finally, a agent for the potential treatment of hypercholesterolaemia and thrombosis has been claimed (Ippen et al, 1983)

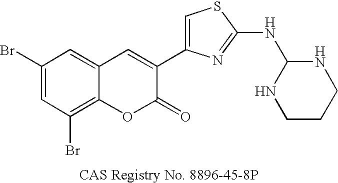

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure.
Owen et al., 2001 “Preparation of hydroxamic acid derivatives as matrix metalloprotease (MMP) inhibitors,” published international (PCT) patent application number WO 01/44189, published 21 Jun. 2001.
Lerchen et al 2001 “Design and optimization of 20-O-linked camptothecin glycoconjugates as anticancer agents.” J. Med. Chem. Vol. 44 No. 22 pp. 4186-4195.
Brown, H D, 1967, “Benzimidazole antihelminthic compositions and methods,” published U.S. Pat. No. 3,338,784, published 29^{th} Aug. 1967.
Orita M et al. 2001, “Coumarin and Chromen-4-one analogues as tautomerase inhibitors of macrophage migration inhibitory factor: discovery and X-ray crystallography.” J. Med. Chem. Vol. 44 No. 4 pp 540-547.
Hlasta D J et al, 1993. “2-substituted saccharin derivative proteolytic enzyme inhibitors,” published European patent application number EP 542372, published 19^{th} May 1993.
Gohar A K, 1990. “Synthesis of several new chromone derivatives and their anti-bacterial activity,” Egypt J. Chem. Vol. 31 No. 3 pp 367-374.
Kalluraya B et al., 2000. “Synthesis and biological activity of 6-substituted-3-[2-(−5-substituted-2-furfurylidenehydrazino)-4-thiazolyl] coumarins.” Boll. Chim. Farm. Vol. 139 No. 6 pp 263-266.
Gursoy, A. et al., 2000. “4-(−3-coumarinyl)-4-thiazolin-2-one benzylidenehydrazones with anti-tuberculosis activity.” Arzneim.-Forsch. Vol. 50 No. 2 pp 167-172.
Kalluraya B et al., 1999. “Synthesis of some triheterocyclic thiazole derivatives of biological interest” Indian J. Heterocycl. Chem. Vol. 8. No. 3 pp. 241-242.
Orme M W et al., 1998 “Compositions and methods for treating bone deficit conditions,” published PCT patent application number WO 9817267, published 30^{th} Apr. 1998.
Petrie C et al., 1997 “Preparation of (hetero)aromatic compounds for treating bone deficit conditions,” published PCT patent application number WO 9715308, published 1^{st} May 1997.
Kalluraya et al, 1995 “Synthesis and biological activity of some 2-aryl-4-coumarinylthiazoles,” Indian J. Heterocycl. Chem. Vol 5 No. 2 pp 153-154.
Yagodinets P I et al., 1995, “Synthesis and antimicrobial activity of derivatives of phosphorus- and nitrogen-containing coumarins.” Khim.-Farm. Zh. Vol. 29 No. 1 pp 49-51.
Gursoy A et al., 1992, “Syntheis and characterization of some 3-acetylcoumarin derivatives and their antimicrobial evaluation” Acta Pharm. Turc. Vol. 34 No. 1 pp 9-15.
Kulkarni Gm et al., 1990. “Synthesis and anti-inflammatory activity of some new triheterocyclic thiazoles”. Rev. Roum. Chem. Vol. 35 No. 4 pp 549-545.
Hishmat O H et al., 1989, “Synthesis of some coumarin derivatives and their antimicrobial activity.” Arch. Pharmacol. Res. Vol. 12 No. 3 pp 181-185.
Chiarino D et al., 1988, “Preparation and formulation of 4-(3-coumarinyl)thiazole derivatives with antiallergic antianaphylactic and antiarthritic activity.” published European patent application number EP 284017, published 28^{th} Sep. 1988.
Hammantgad S S et al., 1984, “Biomimetic thiazolyl coumarins.” Natl. Acad. Sci. Lett. (India) Vol. 7 No. 3 pp 77-78.
Ippen J et al., 1983, “Alkylene-bridged guanidinothiazole derivatives and their use as medicaments” published German patent application number DE 3220118, published 1^{st} Dec. 1983.
Kulkarni M V et al., 1981, “Synthesis and biological properties of some 3-heterocyclic substituted coumarins,” Arch. Pharm. (Weinheim, Ger.) Vol. 34., No. 5 pp 435-439.

### SUMMARY OF THE INVENTION

One aspect of the invention pertains to active compounds, as described herein, which inhibit VEGF production, e.g., in a cell.

Another aspect of the invention pertains to active compounds, as described herein, which inhibit angiogenesis.

Another aspect of the invention pertains to active compounds, as described herein, which treat a proliferative condition, such as cancer.

Another aspect of the present invention pertains to a composition comprising a compound as described herein and a pharmaceutically acceptable carrier.

Another aspect of the present invention pertains to methods of inhibiting VEGF production in a cell, comprising contacting said cell with an effective amount of an active compound, as described herein.

Another aspect of the present invention pertains to methods of inhibiting angiogenesis, comprising contacting a cell with an effective amount of an active compound, as described herein, whether in vitro or in vivo.

Another aspect of the present invention pertains to methods of treating a proliferative condition in a patient comprising administering to said patient a therapeutically-effective amount of an active compound, as described herein. In one preferred embodiment, the proliferative condition is cancer.

Another aspect of the present invention pertains to an active compound, as described herein, for use in a method of treatment of the human or animal body.

Another aspect of the present invention pertains to use of an active compound, as described herein, for the manufacture of a medicament for use in the treatment of a proliferative condition. In one preferred embodiment, the proliferative condition is cancer.

As will be appreciated by one of skill in the art, features and preferred embodiments of one aspect of the invention will also pertain to other aspects of the invention.

### DETAILED DESCRIPTION OF THE INVENTION

The present invention pertains to certain cromen-2-one (coumarin) analogs, specifically to compounds of the formula I

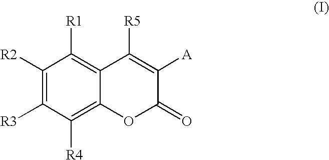

wherein:

A is a four to seven membered heterocyclic ring, aromatic or non aromatic, containing one or more nitrogen, oxygen or sulfur atoms in one or more heterocyclic rings and optionally substituted on the carbon atoms with halogens, alkyls which may be optionally substituted by halogen, amino, hydroxy or cyano groups, aryls, an aromatic or non-aromatic 5- or 6-membered heterocyclic ring containing at least one atom of oxygen, sulfur o niytrogen, hydroxy, amino, monoalkylamino, monoarylamino, bisalkylamino, bisarylamino, (alkyl)(aryl)amino, carbonylamino, alkyl(carbonyl)amino, alkoxycarbonyl, carboxy, cyano groups or, on the nitrogen atoms, with alkyl, aryl, arylalkyl groups or with oxygen atoms to form N-oxides; said four to seven membered heterocyclic ring being optionally fused to one or two aryl, heteroaryl or cycloalkyl groups, in their turn optionally substituted with amino, C₁-C₈-monoalkylamino, monoarylamino, C₁-C₈-bisalkylamino, halogens, alkyl, hydroxy, alkoxycarbonyl, carboxy, cyano groups; said aryl, heteroaryl or cycloalkyl groups being optionally partially saturated or unsaturated, respectively;

R1-R4 are independently selected from:

hydrogen, C₁-C₂₀ alkyl optionally interrupted by one or more heteroatoms such as oxygen, sulfur and nitrogen, hydroxy, C₁-C₈ alkoxy, C₁-C₈ alkoxy optionally substituted with hydroxyl, amino, thio, cyano, carboxy, carboxylic esters or amides, C₁-C₈ haloalkoxy, phenoxy, aralkoxy, C₁-C₈ acyloxy, amino, C₁-C₈ monoalkylamino, C₁-C₈-bisalkylamino, C₁-C₈-acylamino, C₁-C₈-alkylsulfonylamino, aroylamino, halogen, nitro, cyano, trifluoromethyl, carboxy, C₁-C₃ alkoxycarbonyl, a RₐR_{b}N(CH₂)ₙC(═O)— group where Rₐ and R_{b} are independently hydrogen, C₁-C₃-alkyl or Rₐ and R_{b} together with the nitrogen atom they are linked to form a pyrrolidino, piperidino, piperazino or morpholino ring and n=0 or an integer 2 to 4, sulfonyl, mercapto, C₁-C₄-alkylthio, C₁-C₄-alkylsulfonyl, C₁-C₄-alkylsulfinyl, aminosulfonyl, C₁-C₃-alkylaminosulfonyl, a group CH₂NRₐR_{b}, or, taken together with the atoms to which they are attached, R1 and R2 or R2 and R3, or R3 and R4 form an additional aromatic or heteroaromatic ring;

R5 is hydrogen, C₁-C₄-alkyl, C₇-C₁₀ aralkyl,

or a pharmaceutically acceptable salt, solvate, amide, ester, N-oxide, chemically protected form, and prodrug thereof,

as inhibitors of VEGF transcription in mammalian cells.

Examples of heterocyclic rings A are: pyrrolyl, furanyl, thiophenyl, pyrazolyl, thiazolyl, indolyl, oxazolyl, imidazolyl, isothiazolyl, isoxazolyl, 1,2,3-triazolyl, 1,2,4-triazolyl, 1,2,4-oxadiazolyl, 1,3,4-oxadiazolyl, 1,2,5-oxadiazolyl, 1,2,5-thiadiazolyl, 1,3,4-thiadiazolyl, tetrazolyl, pyrimidinyl, pyridazinyle, pyrazinyl, 1,2,4-triazinyl, benzofuranyl, indazolyl, carbazolyl, benzoxazolyl, benziimidazolyl, benzothiazolyl, benzotriazolyl, quinolinyl, isoquinolinyl, cinnolinyl, quinoxalinyl, quinazolinyl, phthalazinyl, 1,2,3-triazinyl, 1,2,4-triazinyl, 1,3,5-triazinyl, purinyl, pteridinyl, benzo[d]imidazo[2,1-b]thiazolyl, 4,5-dihydro-naphtho[1,2-d]thiazolyl, imidazo[1,2-a]pyridinyl.

Preferred meaning for R1, R2, R3, and R4 are hydroxy, C1-C8-alkoxy, amino, C₁-C₈ monoalkylamino, C₁-C₈ bisalkylamino.

Most preferred meanings for R1, R2, R3, and R4 are hydroxy and diethylamino.

Preferred meanings of A are: thiazolyl, 1,3,4-oxadiazolyl, 1,3,4-thiadiazolyl, benzothiazolyl, benzimidazolyl, benzoxazolyl, benzo[d]imidazo[2,1-b]thiazolyl, 4,5-dihydro-naphtho[1,2-d]thiazolyl, imidazo[1,2-a]pyridinyl,

Most particularly preferred meanings of A are thiazolyl, wherein the thiazole ring is connected to the 3-position of the coumarin ring through the 2-, 4- or 5-position, i.e. a 2-thiazolyl, 4-thiazolyl or 5-thiazolyl residue, 1,3,4-oxadiazol-2-yl, 1,3,4-thiadiazol-2-yl, benzothiazol-2-yl, benzimidazol-2-yl, benzoxazol-2-yl,
benzo[d]imidazo[2,1-b]thiazol-2-yl of formula 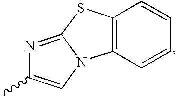
4,5-dihydro-naphtho[1,2-d]thiazole-2-yl of formula

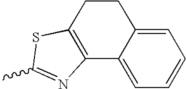

imidazo[1,2-a]pyridine-2-yl of formula

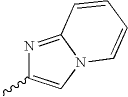

### EXAMPLES OF SPECIFIC EMBODIMENTS

Some individual embodiments of the present invention include the following compounds (Table 1)

TABLE 1

| | | Supplier ID |
|---|---|---|
| # | Structure | (Supplier) |
| 1. | 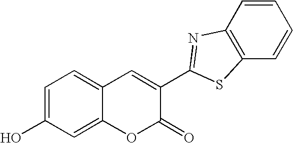 | 12787 (Sigma) |
| 2. | 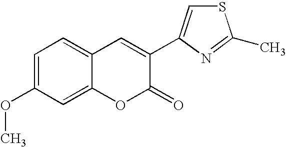 | 1319-0202 (Contact Service) |
| 3. | 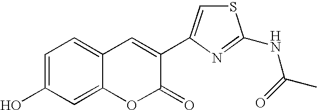 | 1391-0608 (Contact Service) |
| 4. | 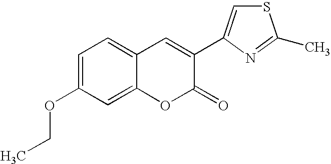 | 1711-1173 (Contact Service) |
| 5. | 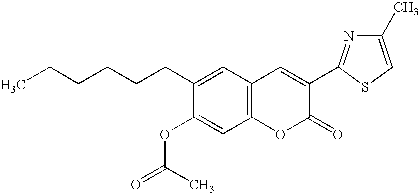 | — |
| 6. | 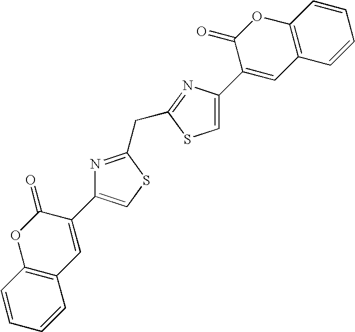 | 0657-0158 (Contact Service) |
| 7. | 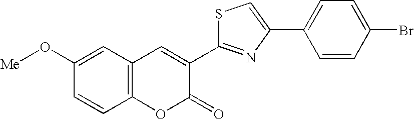 | BAS 1020376 (Asinex) |
| 8. | 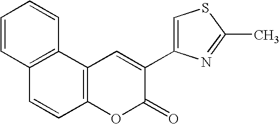 | BAS 1010226 (Asinex) |
| 9. | 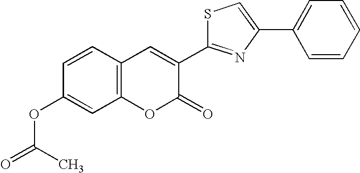 | — |
| 10. | 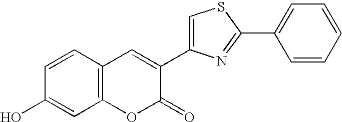 | BAS 1020234 (Asinex) |
| 11. | 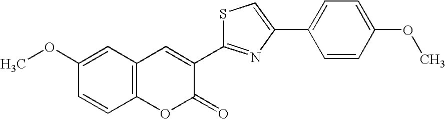 | BAS 1020365 (Asinex) |
| 12. | 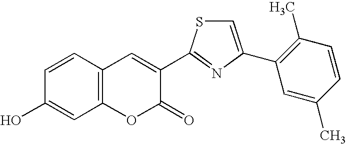 | BAS 1532711 (Asinex) |
| 13. | 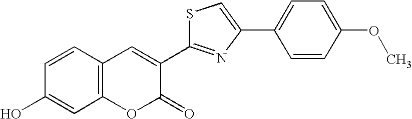 | — |
| 14. | 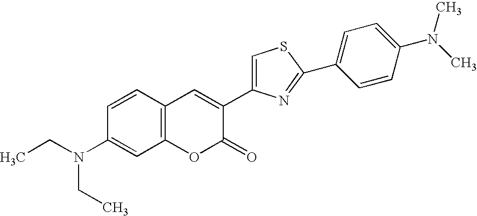 | 0237-0053 (Contact Service) |
| 15. | 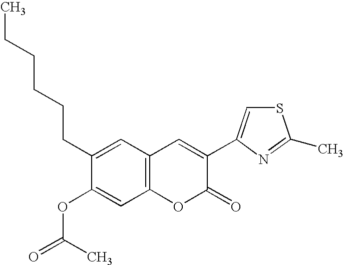 | 0657-0126 (Contact Service) |
| 16. | 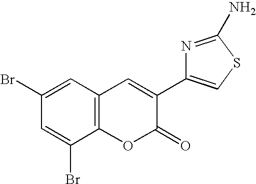 | 1345-2335 (Contact Service) |
| 17. | 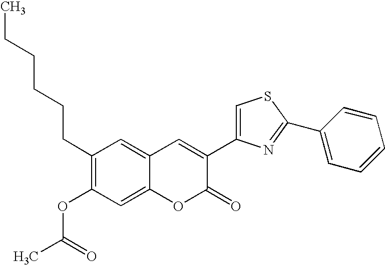 | 2300-3494 (Contact Service) |
| 18. | 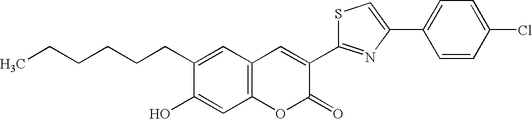 | — |
| 19. | 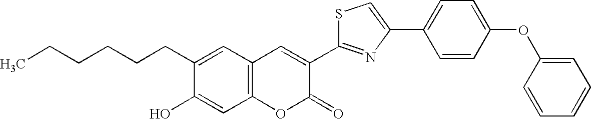 | — |
| 20. | 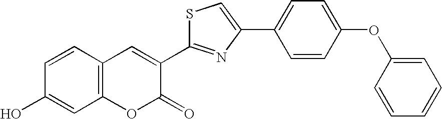 | — |
| 21. | 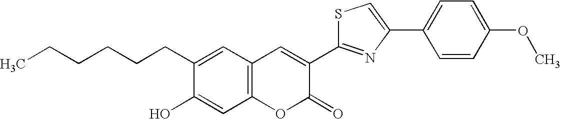 | BAS 1020364 (Asinex) |
| 22. | 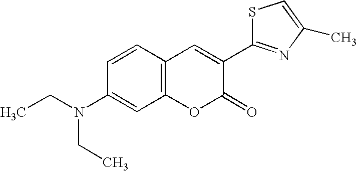 | — |
| 23. | 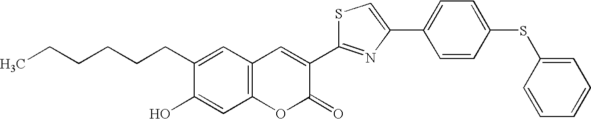 | BAS 1020353 (Asinex) |
| 24. | 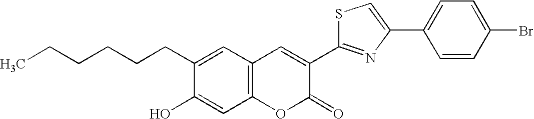 | — |
| 25. | 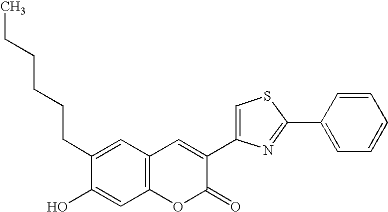 | BAS 1020236 (Asinex) |
| 26. | 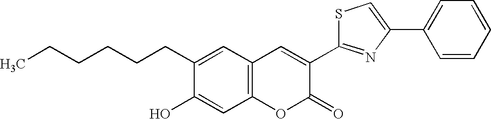 | — |
| 27. | 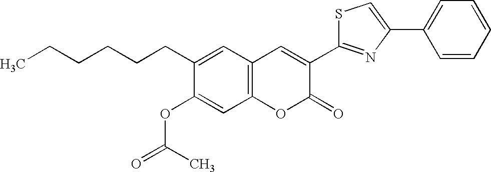 | BAS 1020292 (Asinex) |
| 28. | 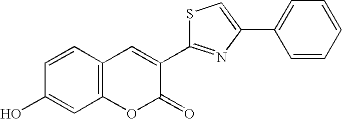 | — |
| 29. | 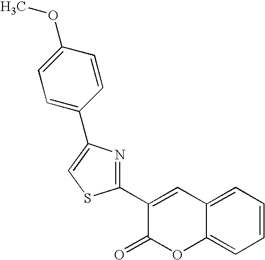 | BAS 1235397 (Asinex) |
| 30. | 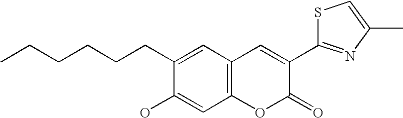 | — |
| 31. | 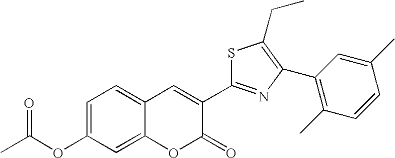 | BAS 1922929 (Asinex) |
| 32. | 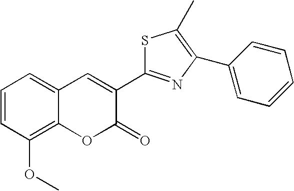 | BAS 2233269 (Asinex) |
| 33. | 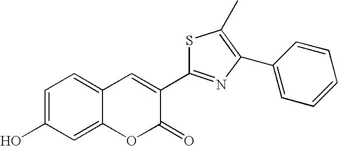 | BAS 2233281 (Asinex) |
| 34. | 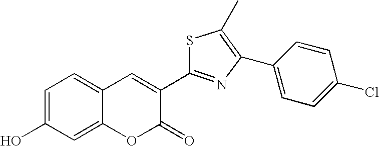 | BAS 2233282 (Asinex) |
| 35. | 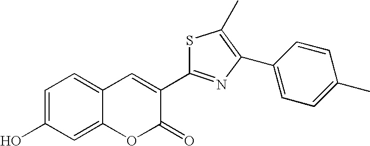 | BAS 2233285 (Asinex) |
| 36. | 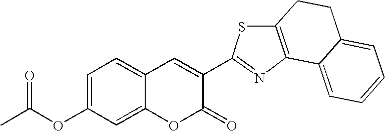 | BAS 2171780 (Asinex) |
| 37. | 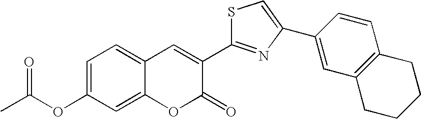 | BAS 2171807 (Asinex) |
| 38. | 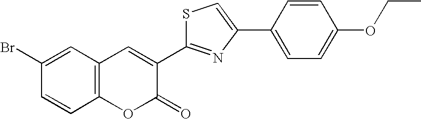 | 1246-0825 (Contact Service) |
| 39. | 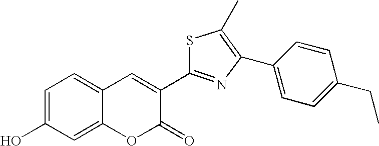 | BAS 2233286 (Asinex) |
| 40. | 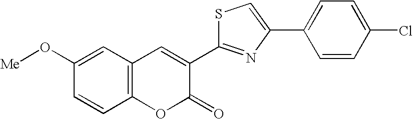 | BAS 1020387 (Asinex) |
| 41. | 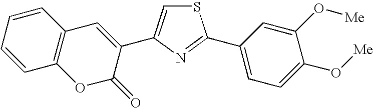 | BAS 3014799 (Asinex) |
| 42. | 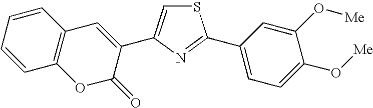 | BAS 1922916 (Asinex) |
| 43. | 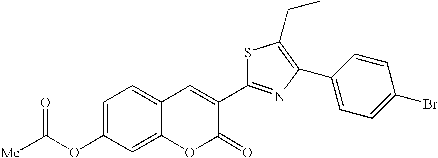 | BAS 1922924 (Asinex) |
| 44. | 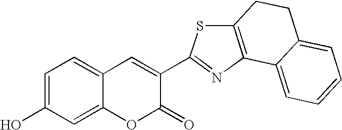 | BAS 2171779 (Asinex) |
| 45. | 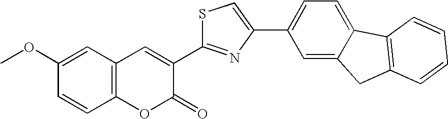 | BAS 1020319 (Asinex) |
| 46. | 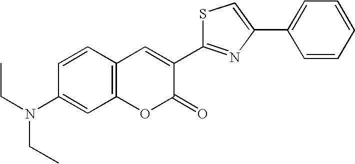 | BAS 1020295 (Asinex) |
| 47. | 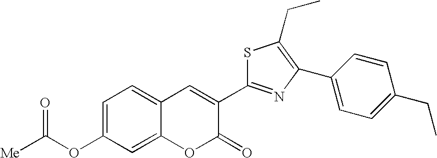 | BAS 1922927 (Asinex) |
| 48. | 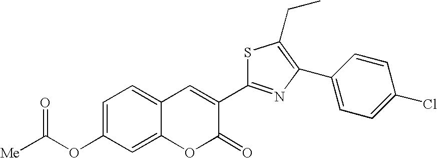 | BAS 1922923 (Asinex) |
| 49. | 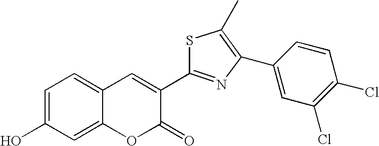 | BAS 2233344 (Asinex) |
| 50. | 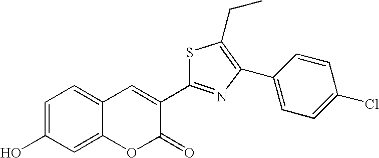 | BAS 1922915 (Asinex) |
| 51. | 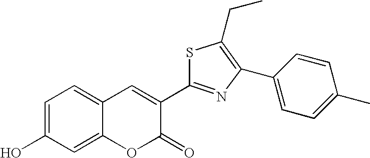 | BAS 1922918 (Asinex) |
| 52. | 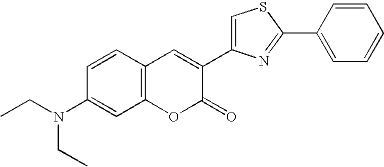 | BAS 1020238 (Asinex) |
| 53. | 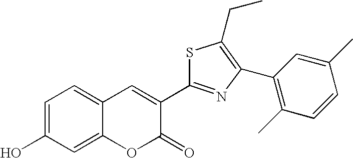 | BAS 1922921 (Asinex) |
| 54. | 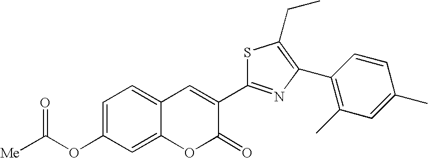 | BAS 1922928 (Asinex) |
| 55. | 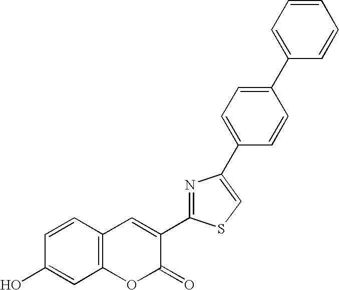 | BAS 1020302 (Asinex) |
| 56. | 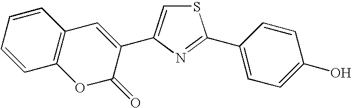 | BAS 3014798 (Asinex) |
| 57. | 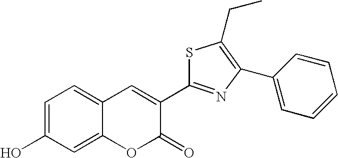 | BAS 1922914 (Asinex) |
| 58. | 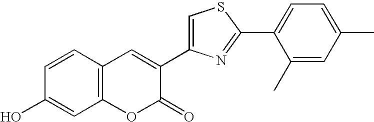 | 5349533 (Chembridge) |
| 59. | 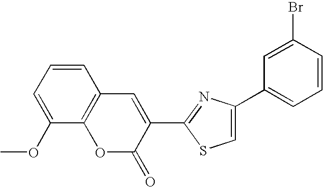 | 5941684 (Chembridge) |
| 60. | 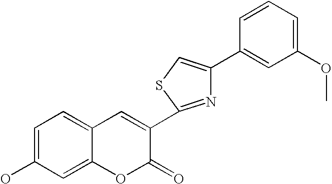 | — |
| 61. | 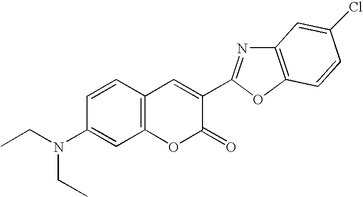 | 6125876 (Chembridge) |
| 62. | 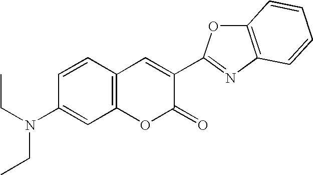 | BAS 0212730 (Asinex) |
| 63. | 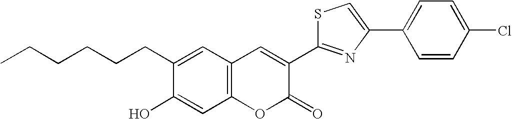 | BAS 1020312 (Asinex) |
| 64. | 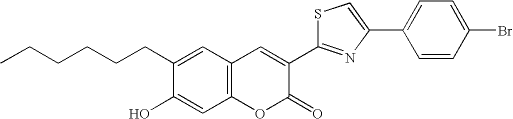 | BAS 1020382 (Asinex) |
| 65. | 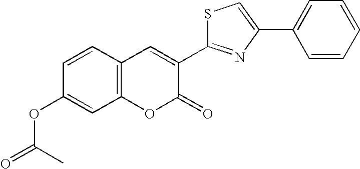 | — |
| 66. | 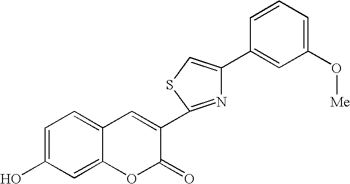 | — |
| 67. | 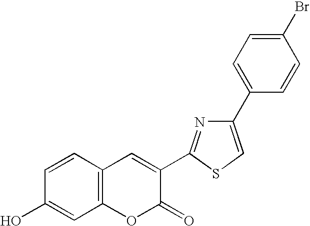 | — |
| 68. | 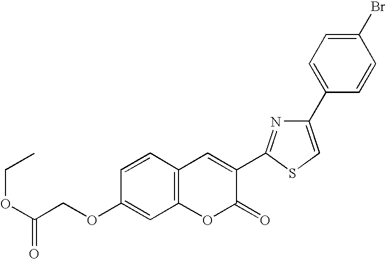 | — |
| 69. | 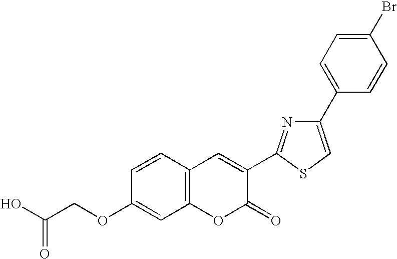 | — |
| 70. | 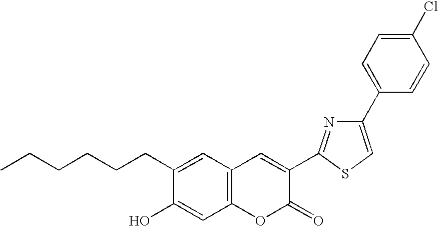 | — |
| 71. | 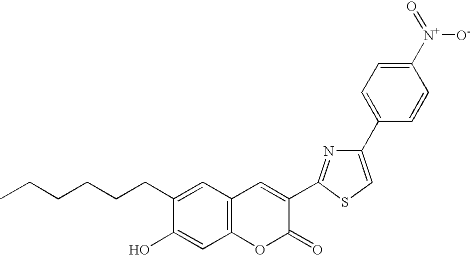 | — |
| 72. | 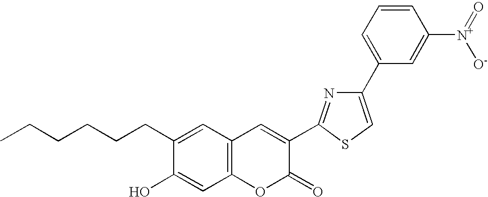 | — |
| 73. | 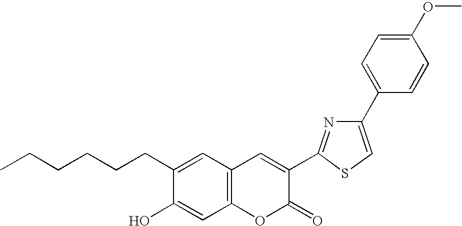 | — |
| 74. | 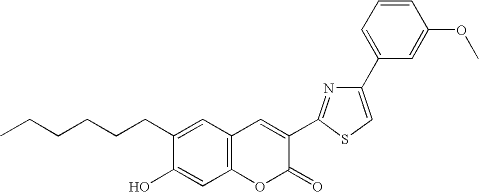 | — |
| 75. | 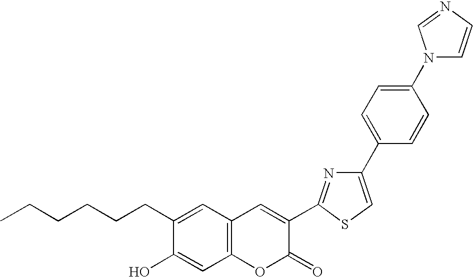 | — |
| 76. | 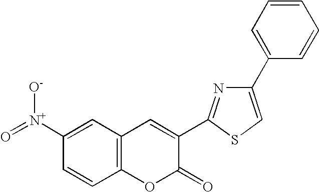 | — |
| 77. | 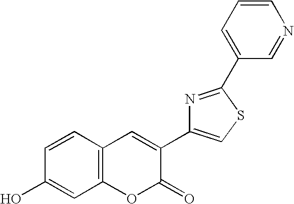 | — |
| 78. | 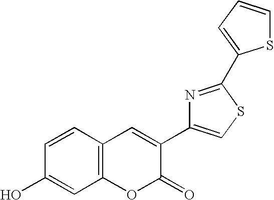 | — |
| 79. | 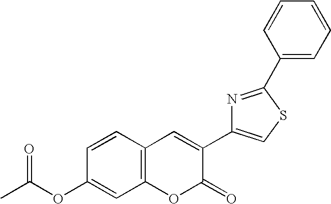 | — |
| 80. | 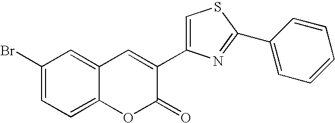 | — |
| 81. | 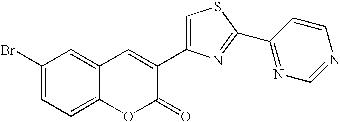 | — |
| 82. | 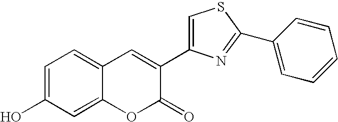 | — |
| 83. | 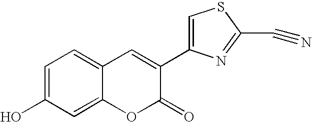 | — |
| 84. | 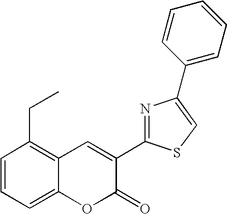 | — |
| 85. | 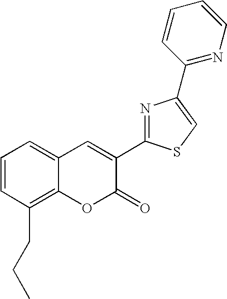 | — |
| 86. | 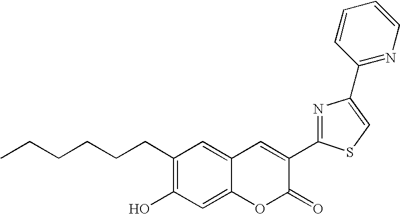 | — |
| 87. | 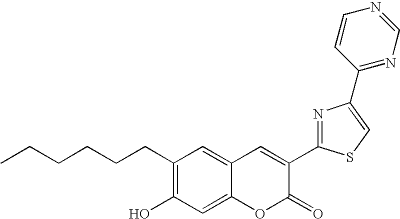 | — |
| 88. | 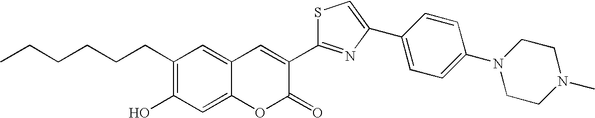 | — |
| 89. | 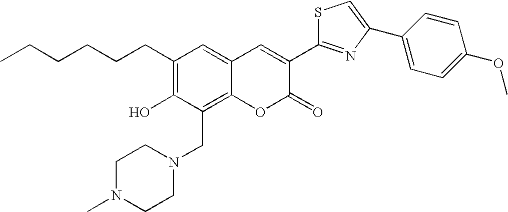 | — |
| 90. | 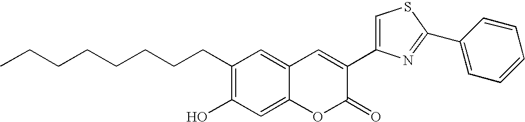 | — |
| 91. | 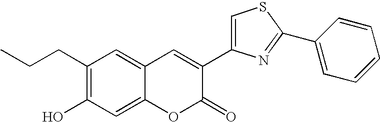 | — |
| 92. | 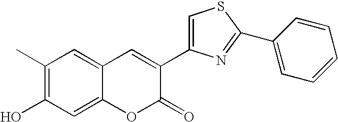 | — |
| 93. | 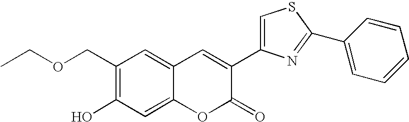 | — |
| 94. | 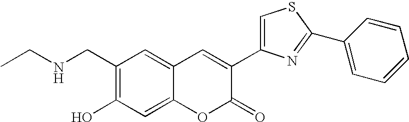 | — |
| 95. | 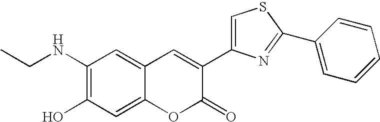 | — |
| 96. | 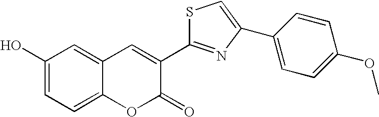 | — |
| 97. | 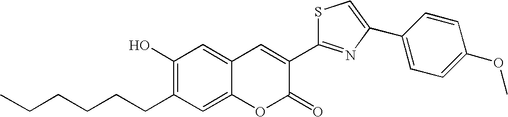 | — |
| 98. | 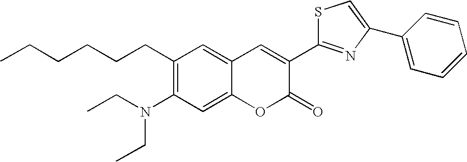 | — |
| 99. | 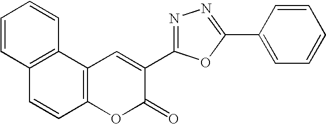 | 0657-0057 (Contact Service) |
| 100. | 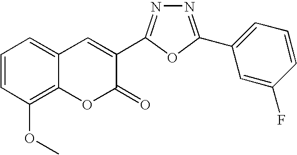 | BAS 1289938 (Asinex) |
| 101. | 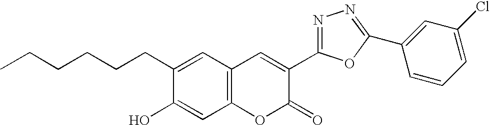 | BAS 1289947 (Asinex) |
| 102. | 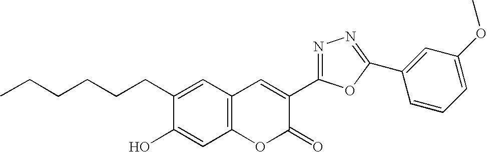 | BAS 1289945 (Asinex) |
| 103. | 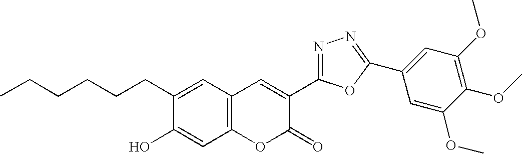 | BAS 1289951 (Asinex) |
| 104. | 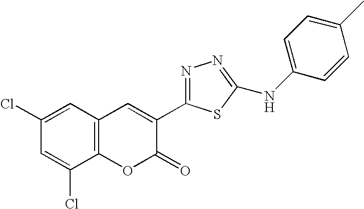 | BAS 1290020 (Asinex) |
| 105. | 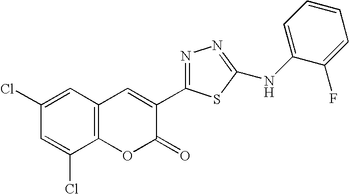 | BAS 1290021 (Asinex) |
| 106. | 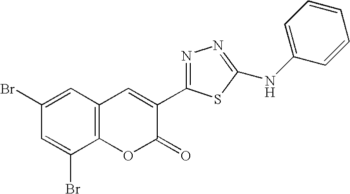 | BAS 1290017 (Asinex) |
| 107. | 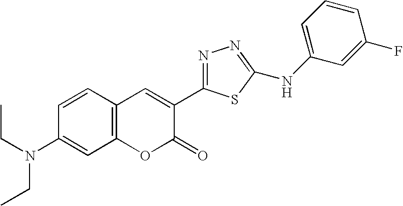 | BAS 1290025 (Asinex) |
| 108. | 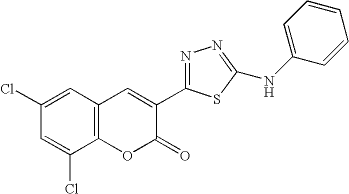 | BAS 1290018 (Asinex) |
| 109. | 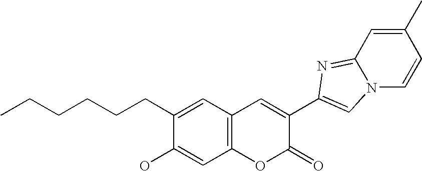 | BAS 1290079 (Asinex) |
| 110. | 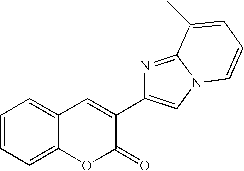 | BAS 1290067 (Asinex) |
| 111. | 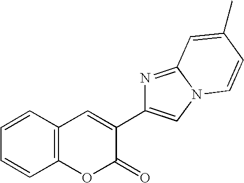 | BAS 1290074 (Asinex) |
| 112. | 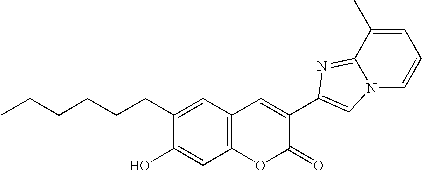 | BAS 1290072 (Asinex) |
| 113. | 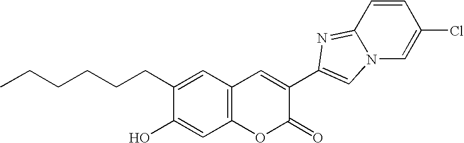 | BAS 1290066 (Asinex) |
| 114. | 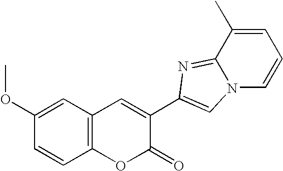 | BAS 1290068 (Asinex) |
| 115. | 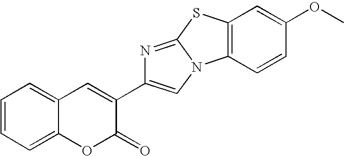 | BAS 1290095 (Asinex) |
| 116. | 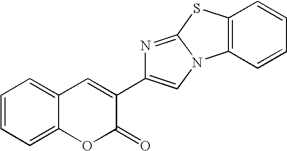 | BAS 1290086 (Asinex) |
| 117. | 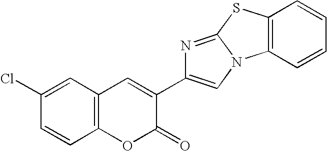 | BAS 1290103 (Asinex) |
| 118. | 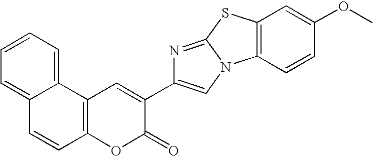 | BAS 1290096 (Asinex) |
| 119. | 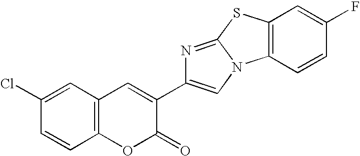 | BAS 1290101 (Asinex) |
| 120. | 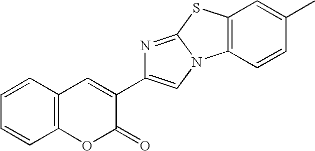 | BAS 1290090 (Asinex) |
| 121. | 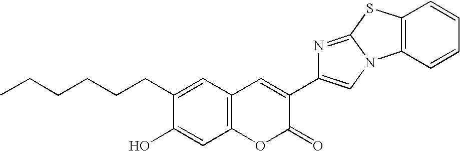 | BAS 1290087 (Asinex) |
| 122. | 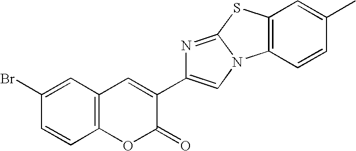 | BAS 1290092 (Asinex) |
| 123. | 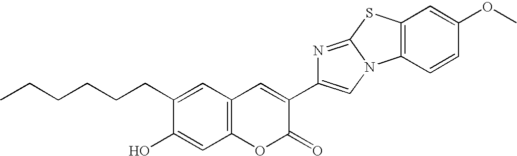 | BAS 1290098 (Asinex) |
| 124. | 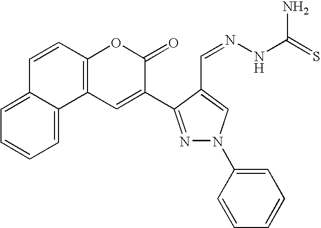 | 5916661 (Chembridge) |
| 125. | 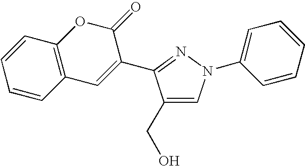 | 6527141 (Chembridge) |
| 126. | 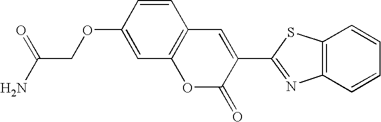 | AE-641/15338368 (Specs) |
| 127. | 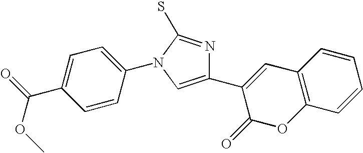 | AF-399/40882594 (Specs) |
| 128. | 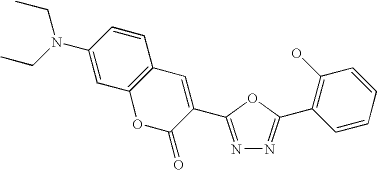 | AG-690/12699018 (Specs) |
| 129. | 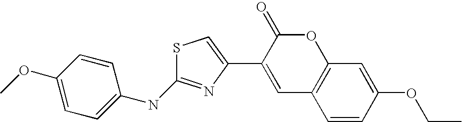 | AG-690/13505023 (Specs) |
| 130. | 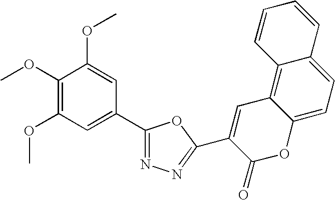 | AG-690/13507052 (Specs) |
| 131. | 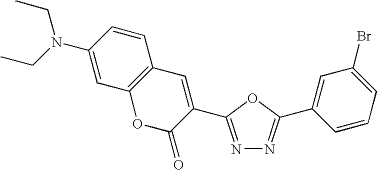 | AG-690/13507054 (Specs) |
| 132. | 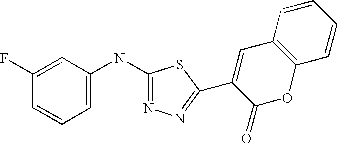 | AG-690/13507089 (Specs) |
| 133. | 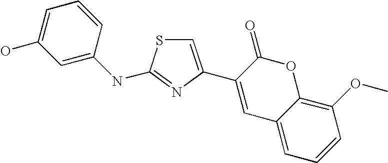 | AP-263/40917434 (Specs) |
| 134. | 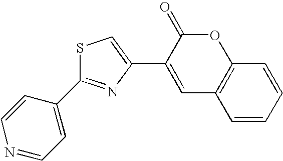 | AP-048/15613073 (Specs) |
| 135. | 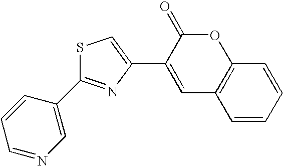 | AP-048/15614133 (Specs) |
| 136. | 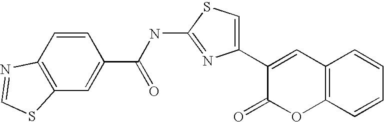 | F0537-0272 (IFLABS) |
| 137. | 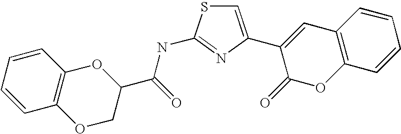 | F0537-0271 (IFLABS) |
| 138. | 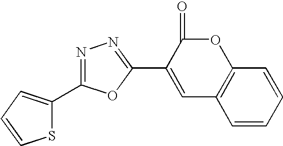 | F0777-2151 (IFLABS) |
| 139. | 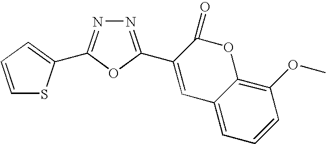 | F0777-2150 (IFLABS) |
| 140. | 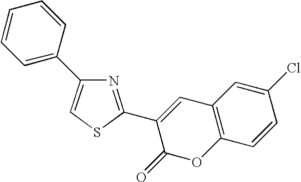 | F0913-3352 (IFLABS) |
| 141. | 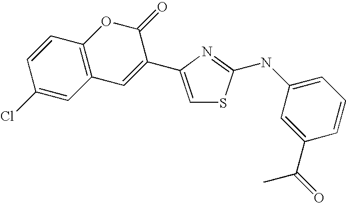 | F0913-3345 (IFLABS) |
| 142. | 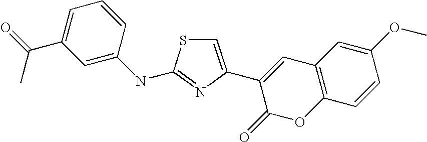 | F0913-3343 (IFLABS) |
| 143. | 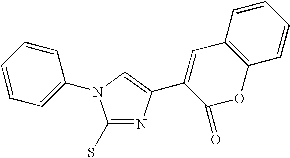 | F1045-0014 (IFLABS) |
| 144. | 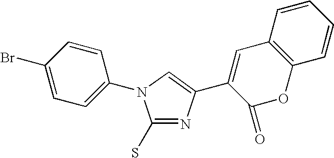 | F1045-0011 (IFLABS) |
| 145. | 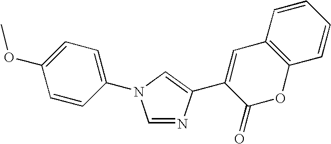 | F1045-0010 (IFLABS) |
| 146. | 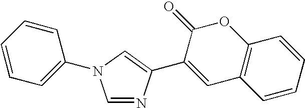 | F1045-0009 (IFLABS) |
| 147. | 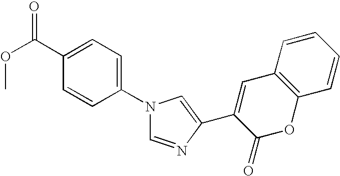 | F1045-0008 (IFLABS) |
| 148. | 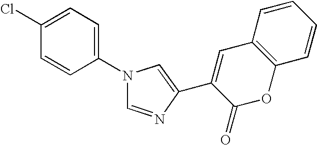 | F1045-0007 (IFLABS) |
| 149. | 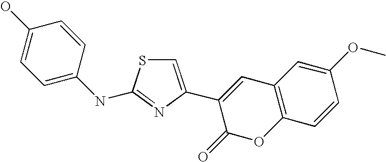 | F1053-0429 (IFLABS) |
| 150. | 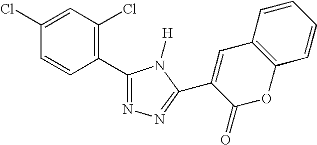 | F1058-0449 (IFLABS) |
| 151. | 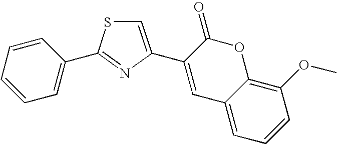 | F1107-0224 (IFLABS) |
| 152. | 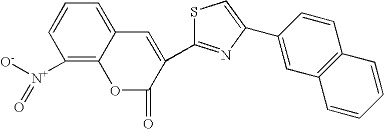 | F1110-0453 (IFLABS) |
| 153. | 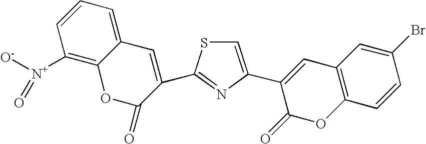 | F1110-0451 (IFLABS) |
| 154. | 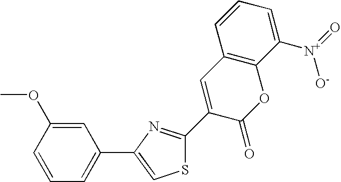 | F1110-0444 (IFLABS) |
| 155. | 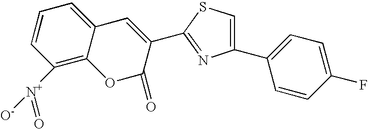 | F1110-0431 (IFLABS) |
| 156. | 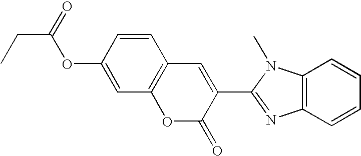 | F1190-0433 (IFLABS) |
| 157. | 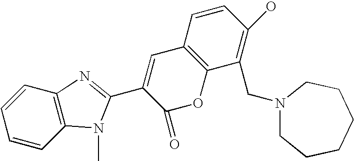 | F1190-0076 (IFLABS) |
| 158. | 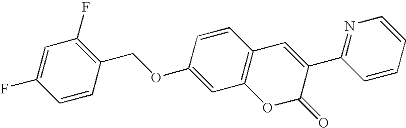 | F1190-0043 (IFLABS) |
| 159. | 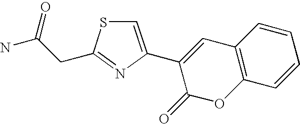 | F1194-0013 (IFLABS) |
| 160. | 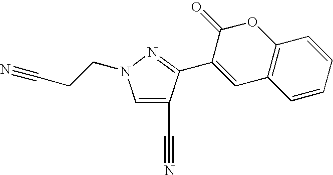 | F1218-1056 (IFLABS) |

Synthesis

The compounds of the present invention may be prepared using well known methods, or by adapting well known methods in well known ways.

For example, the compounds of the invention featuring a thiazolyl residue (2-thiazolyl or 4-thiazolyl) attached at the 3 position of the coumarin ring may be prepared according to the schemes 1 and 2 and 3 reported in the example-section.

Some compounds of the present invention are available from commercial sources, such as Chembridge, Specs, Iflabs.

Uses

The present invention provides active compounds which are capable of inhibiting the production of VEGF, as well as methods of inhibiting VEGF production, comprising contacting a cell with an effective amount of an active compound, whether in vitro or in vivo.

The term “active,” as used herein, pertains to compounds which are capable of inhibiting VEGF production, and specifically includes both compounds with intrinsic activity (drugs) as well as prodrugs of such compounds, which prodrugs may themselves exhibit little or no intrinsic activity.

One of ordinary skill in the art is readily able to determine whether or not a candidate compound is active, that is, capable of inhibiting VEGF production, for example, capable of inhibiting the transcription of the VEGF gene. For example, assays which may conveniently be used to assess the inhibition offered by a particular compound are described in the examples below.

For example, a sample of cells (e.g., from a tumour) may be grown in vitro and a candidate compound brought into contact with the cells, and the effect of the compound on those cells observed. As examples of “effect”, the expression levels of the VEGF gene may be determined. Where the candidate compound is found to exert an influence on the cells, this may be used as a prognostic or diagnostic marker of the efficacy of the compound in methods of treating a patient carrying the tumour or a tumour of the same cellular type.

Thus, in one aspect, the present invention provides angiogenesis inhibitors, as well as methods of inhibiting angiogenesis, comprising contacting a cell (e.g., a tumour cell, an endothelial cell, etc.) with an effective amount of an active compound, whether in vitro or in vivo. The term “angiogenesis inhibitor” as used herein, pertains to an active compound which inhibits angiogenesis, that is, which inhibits the progress of angiogenesis, and includes both a reduction in the rate of progress and a halt in the rate of progress.

Thus, in one aspect, the present invention provides antiproliferative agents. The term “antiproliferative agent” as used herein, pertain to a compound which treats a proliferative condition (i.e., a compound which is useful in the treatment of a proliferative condition).

The terms “cell proliferation,” “proliferative condition,” “proliferative disorder,” and “proliferative disease,” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

Antiproliferative compounds of the present invention have application in the treatment of cancer, and so the present invention further provides anticancer agents. The term “anticancer agent” as used herein, pertains to a compound which treats a cancer (i.e., a compound which is useful in the treatment of a cancer). The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The active compounds of the present invention are particularly applicable to proliferative conditions (e.g., cancers) which are characterized by so-called “solid” tumours, and which rely on angiogenesis, and the vasculature arising therefrom.

The invention further provides active compounds for use in a method of treatment of the human or animal body. Such a method may comprise administering to such a subject a therapeutically-effective amount of an active compound, preferably in the form of a pharmaceutical composition.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio.

The invention further provides the use of an active compound for the manufacture of a medicament, for example, for the treatment of a proliferative condition, as discussed above.

The invention further provides a method of treatment of the human or animal body, the method comprising administering to a subject in need of treatment a therapeutically-effective amount of an active compound, preferably in the form of a pharmaceutical composition.

Active compounds may also be used, as described above, in combination therapies, that is, in conjunction with other agents, for example, cytotoxic agents.

Active compounds may also be used as part of an in vitro assay, for example, in order to determine whether a candidate host is likely to benefit from treatment with the compound in question.

Active compounds may also be used as a standard, for example, in an assay, in order to identify other active compounds, other antiproliferative agents, etc.

Administration

The active compound or pharmaceutical composition comprising the active compound may be administered to a subject by any convenient route of administration, whether systemically/peripherally or at the site of desired action, including but not limited to, oral (e.g, by ingestion); topical (including transdermal, intranasal, ocular, buccal, and sublingual); pulmonary (e.g., by inhalation therapy using, for example, an aerosol); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal.

The subject may be a eukaryote, an animal, a vertebrate animal, a mammal, a rodent (e.g., a guinea pig, a hamster, a rat, a mouse), murine (e.g., a mouse), a simian (e.g., a chimpanzee), or a human.

Formulations

While it is possible for the active ingredient to be administered alone, it is preferable to present it as a pharmaceutical composition (e.g., formulation) comprising at least one active ingredient, as defined above, together with one or more pharmaceutically acceptable carriers, excipients, buffers, adjuvants, stabilisers, or other materials well known to those skilled in the art and optionally other therapeutic agents.

Thus, the present invention further provides pharmaceutical compositions, as defined above, and methods of making a pharmaceutical composition comprising admixing at least one active ingredient, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

Formulations may be in the form of liquids, solutions, suspensions, emulsions, tablets, losenges, granules, powders, capsules, cachets, pills, ampoules, suppositories, pessaries, ointments, gels, pastes, creams, sprays, foams, lotions, oils, boluses, electuaries, or aerosols.

Formulations suitable for oral administration (e.g., by ingestion) may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose), surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active ingredients and optionally one or more excipients or diluents.

Formulations suitable for topical administration in the mouth include losenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active ingredient.

Formulations suitable for topical administration via the skin include ointments, creams, and emulsions. When formulated in an ointment, the active ingredient may optionally be employed with either a paraffinic or a water-miscible ointment base. Alternatively, the active ingredients may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active ingredient through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at lease one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active ingredient in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freese-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect.

Administration in vivo can be effected in one dose, continuously or intermittently throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 0.1 to about 250 mg per kilogram body weight of the subject per day. Where the active ingredient is a salt, an ester, prodrug, or the like, the amount administered is calculated on the basis the parent compound and so the actual weight to be used is increased proportionately.

### EXAMPLES

The following examples are provided solely to illustrate the present invention and are not intended to limit the scope of the invention, as described herein.

The compounds of the invention featuring a thiazolyl residue (2-thiazolyl or 4-thiazolyl) attached at the 3 position of the coumarin ring may be prepared according to the following schemes 1 and 2.

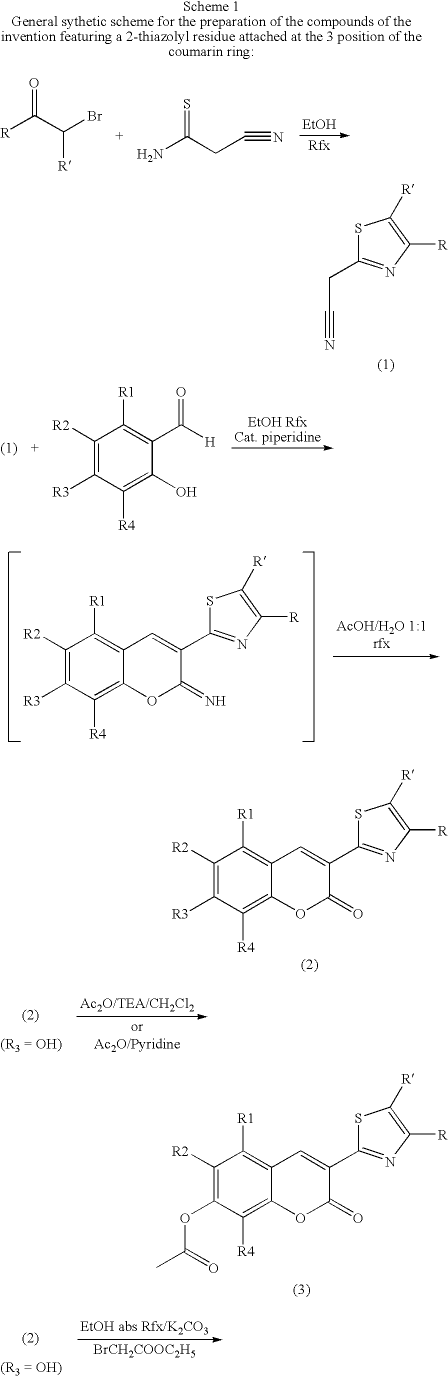

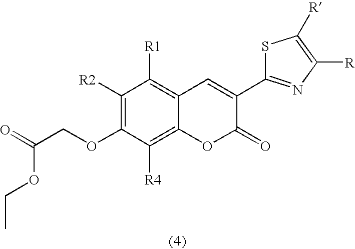

Compounds of Formula (1)

Commercially available α-cyanothioacetamide is dissolved in hot DMF or EtOH (0.2 g/in L; 50-60° C.) and the stoichiometric amount of α-bromoketone, dissolved in DMF or EtOH (0.5 g/mL), is added dropwise over a period of about 30′.

After the addition is completed, the mixture is stirred overnight at room temperature.

In process control: TLC (SiO₂; hexane/AcOEt 8:2).

The mixture is poured into water and extracted with AcOEt. The combined organic phases are then washed with brine and dried over Na₂SO₄. After concentration under vacuum the oily residue is chromatographed on a silica gel column (hexane/AcOEt 9:1, SiO₂ 1:25) to yield (usually around 80%) pure [4-substituted-thiazol-2-yl] acetonitrile.

Analytical control: TLC, melting point, Elemental analysis, 1H-NMR (CDCl₃ or DMSO-d₆).

Note:

1. Whereas the α-bromoketone is not commercially available it can be readly prepared following the procedure described for compounds of formula (5) in Scheme 2.

Compounds of Formula (2) Method A

An example of this route of synthesis, without experimental details, is described in J. Chem. Research (S) 1997, 240-241.

The suitable salicylaldehyde² (0.05 g/mL) is mixed in absolute Ethanol together with the above prepared [4-substituted-thiazol-2-yl] acetonitrile (stoichiometric amount, 0.05 g/mL) and the resultant mixture heated under reflux. Then a few drops of piperidine are added and the mixture is stirred for about 1 hour. A solid generally precipitates. After cooling at room temperature the precipitated solid, the intermediate imino derivative³, is filtered off, washed with EtOH and dried under vacuum (2 h, 60° C.).

In process control: TLC (SiO₂; hexane/AcOEt 1:1).

The isolated intermediate is then suspended in AcOH/H₂O 1:1 (0.04 g/mL) and the mixture refluxed for 4 hours. The suspension is cooled to room temperature and the solid removed by filtration and washed several times with water. After drying under vacuum (5 h, 60° C.; overnight, 25° C.) the yield is generally around 70%.

Analytical control: TLC, melting point, Elemental analysis, ¹H-NMR (DMSO-d₆).

Note:

2. The 4-hydroxysalicylaldehyde and the 4-diethylaminosalicylaldehyde are commercially available whereas the 5-hexyl-4-hydroxysalicylaldehyde and the 5-ethyl-4-hydroxysalicylaldehyde were readily prepared from, respectively, 4-hexylresorcinol and 4-ethylresorcinol according to a literature reference: J. Med. Chem. 1998, 41, 4819-4832.

3. When the starting material is 4-diethylaminosalicylaldehyde, the imino-derivative intermediate is an oily residue obtained by concentrating the reaction mixture. This oily residue is then suspended in AcOH/H₂O 1:1 as in the above procedure.

Compounds of Formula (2) Method B

Commercially available α-cyanothioacetamide is suspended in glacial acetic acid (0.033 g/mL) along with the stoichiometric amount of α-bromoketone and sodium acetate. The mixture is refluxed for 1 hour then cooled to room temperature. The suitable salicylaldehyde (0.033 g/mL) is added and the mixture refluxed for 16 hours. An additional amount of salicylaldehyde (0.017 g/mL) is added and the mixture refluxed for 8 hours then cooled. The solid is removed by filtration, washed with a 1:1 mixture of EtOH:Et₂O and resuspended in acetic acid containing 10% of water (0.033 g/mL). The mixture is refluxed for 1 hour then cooled to room temperature and the solid removed by filtration and washed several times with 9:1 acetic acid:water mixture. After drying under vacuum (5 h, 40° C.; overnight, 25° C.) the yield is generally around 53%.

Analytical control: TLC, melting point, Elemental analysis, ¹H-NMR (DMSO-d₆).

Compounds of Formula (2) Method C

A compound of formula (12) (see below, Scheme 3) is suspended in ethanol (0.025 g/mL) along with a stoichiometric amount of a suitable α-bromoketone. The mixture is refluxed for 5.5 hours then cooled to room temperature. The solid is collected and dissolved in CH₂Cl₂. The solution is filtered on a silica gel column and evaporated to dryness under vacuum. The solid residue is collected and triturated with Prⁱ₂/EtOH. The yield is generally around 42%.

Analytical control: TLC, Elemental analysis, ¹H-NMR (DMSO-d₆).

Compounds of Formula (3)

The suitable compound (2) (with R₃═OH) is dissolved (sometimes incompletely) in anhydrous pyridine (0.1 g/mL) and the solution is cooled at 0-5° C. Then acetic anhydride (10% molar excess) is added dropwise and a precipitate is readily formed. After 1 hour at 0-5° C. and further 1 hour at room temperature the reaction is complete as judged by TLC (SiO₂; hexane/AcOEt 8:2).

The precipitated solid is washed with hexane and dried under vacuum (3 h, 40° C.). The yield is usually around 50-60%.

Analytical control: TLC, Elemental analysis, ¹H-NMR (DMSO-d₆).

Compounds of Formula (4)

The suitable compound (2) (with R₃═OH) (0.016 g/mL) is mixed in absolute Ethanol together with K₂CO₃ (stoichiometric amount, 0.016 g/mL), Ethyl Bromoacetate (stoichiometric amount, 0.016 g/mL) and the resultant mixture heated under reflux for 8 hours. Additional Ethyl Bromoacetate (stoichiometric amount, 0.016 g/mL) is added and the mixture further refluxed for 4 hours.

The suspension is cooled to room temperature and the solid removed by filtration and crystallyzed from DMF. The crystallyzed solid is filtered off, washed with cold DMF and dried under vacuum (40° C.).

The yield is usually around 50-60%.

Analytical control: TLC, Elemental analysis, ¹H-NMR (DMSO-d₆).

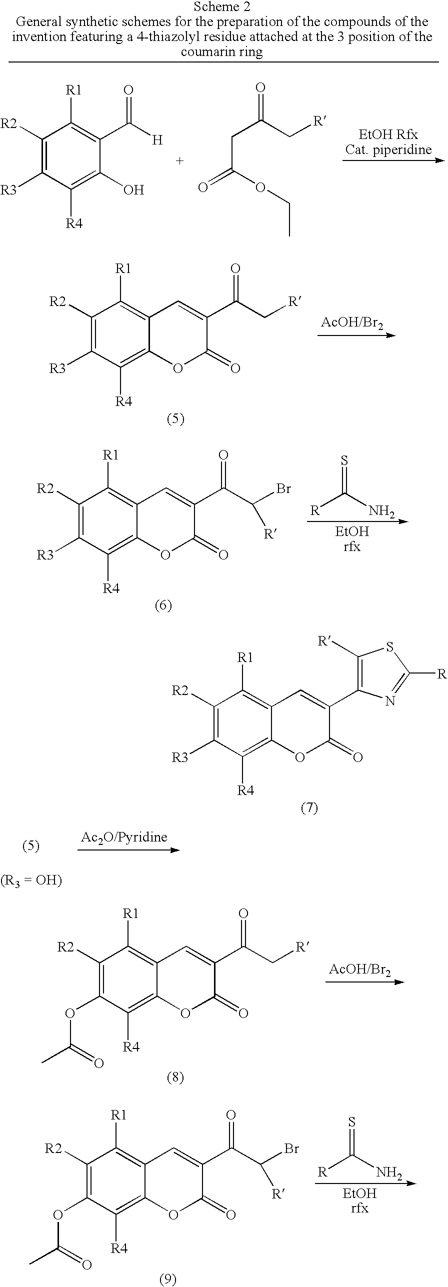

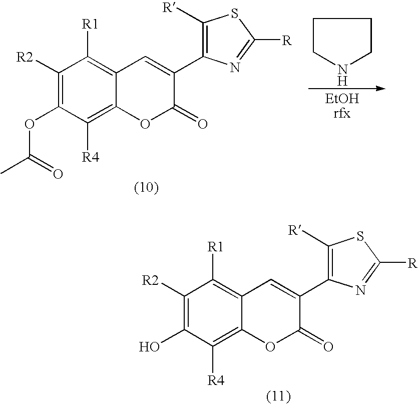

Compounds of Formula (5)

The suitable salicylaldehyde1 (0.33 g/mL) is mixed in absolute Ethanol together with commercially available ethyl acetoacetate (stoichiometric amount, 0.5 g/mL), a catalytic amount of piperidine and the resultant mixture heated at 50° C. for about 3 hours. A solid generally precipitates. After cooling at room temperature the precipitated solid is filtered off, washed with EtOH and Hexane and dried under vacuum (2 h, 60° C.).

The yield is generally around 65-80%.

In process control: TLC (SiO₂; hexane/AcOEt 1:1).

Analytical control: TLC, melting point, Elemental analysis, 1H-NMR (DMSO-d₆).

Compounds of Formula (6)

The suitable compound (5) is suspended in glacial Acetic Acid (0.32 g/mL) and the mixture cooled to 10° C. A stoichiometric amount of neat bromine is added dropwise and the mixture stirred at room temperature for about 2.30 h. The colour of the mixture turns from red to pale yellow and a solid generally precipitates. The precipitated solid is filtered off, washed with AcOEt and Hexane and dried under vacuum (2 h, 60° C.).

In process control: TLC (SiO₂; hexane/AcOEt 1:1).

The yield is generally around 75%.

Analytical control: TLC, melting point, Elemental analysis, 1H-NMR (CDCl3 or DMSO-d₆).

Compounds of Formula (7)

The above prepared compound (6) is suspended in hot EtOH (0.015 g/mL; 80° C.) and a stoichiometric amount of commercially available thiocarboxamide is added. After initial dissolution of the suspended reagents, the solution turns yellow and a solid generally precipitates. After two hours at 80° C. the mixture is cooled and the precipitated solid is filtered off, washed with EtOH and dried under vacuum (2 h, 60° C.).

In process control: TLC (SiO₂; hexane/AcOEt 1:1).

The yield is generally around 65%.

Analytical control: TLC, melting point, Elemental analysis, ¹H-NMR (CDCl₃ or DMSO-d₆).

Compounds of Formula (8)

The suitable compound (5) (with R3=OH) is suspended in anhydrous pyridine (0.11 g/mL) and acetic anhydride (2.5 molar excess) is added dropwise. A precipitate is readily formed. After 4 hour at room temperature the precipitated solid is washed with hexane and dried under vacuum (3 h, 40° C.). The yield is usually around 50-60%.

TLC (SiO₂; hexane/AcOEt 1:1).

The yield is generally around 55-60%.

Analytical control: TLC, Elemental analysis, 1H-NMR (DMSO-d₆).

Compounds of Formula (9)

Starting from the suitable compound (8) and following the procedure described for compound (6), compound (9) is obtained as a solid.

TLC (SiO₂; hexane/AcOEt 1:1).

The yield is generally around 70%.

Analytical control: TLC, Elemental analysis, 1H-NMR (DMSO-d₆).

Compounds of Formula (10)

Starting from the suitable compound (9) and following the procure described for compound (7), a mixture of compound (10) and compound (11) is obtained.

TLC (SiO₂; hexane/AcOEt 1:1).

Analytical control: TLC, 1H-NMR (DMSO-d₆).

Compounds of Formula (11)

The above prepared⁴ mixture of compounds (10) and (11) is suspended in EtOH (0.1 g/mL) and pyrrolidine (10 times excess) is added while stirring. After 30 min the reaction is completed. In process control: TLC (SiO₂; hexane/AcOEt 1:1).

Following adjunction of 2 N HCl up to pH=6, a solid generally precipitates. Water is added (5:1, vs EtOH) and the mixture is stirred for 10 min.

The precipitated solid is filtered off, washed with water and dried under vacuum (2 h, 50° C.)

The yield is generally around 90%.

Analytical control: TLC, melting point, Elemental analysis, 1H-NMR (CDCl₃ or DMSO-d₆).

Note:

4. When the thiocarboxamide used for preparation of compound (10) contains a basic nitrogen there is no precipitation of solid material. The mixture is evaporated under vacuum and the crude material is suspended in water (0.025 g/mL), treated with cold 0.1 N HCl up to pH=2 and is stirred for 10 min at room temperature. The solid is filtered off, washed with brine and dried under vacuum (2 h, 50° C.). The compound is obtained as hydrochloride.

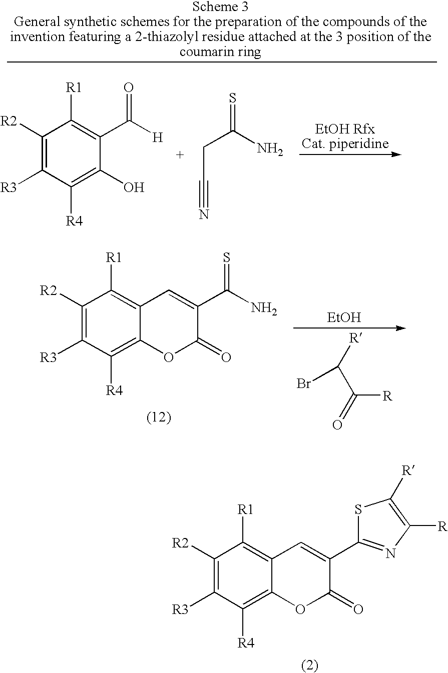

Compounds of Formula (12)

To a solution of a suitable salicylaldehyde (0.019 g/mL) and a stoichiometric amount of commercially available 2-cyanothioacetamide in absolute EtOH are added a few drops of piperidine. The resulting solution is then stirred for 24 hours at room temperature. A solid generally precipitates. The precipitated solid is collected, washed with absolute EtOH and dried under vacuum. The yield is generally around 90%.

Analytical control: TLC, ¹H-NMR (DMSO-d₆).

Specific Synthesis of the Compounds of the Invention

Synthesis of Intermediates

Description 1.5-hexyl-4-hydroxysalicylaldehyde

According to the described procedure (J. Med. Chem., 41, 24, 4819-4832, 1998) the synthesis of the title compound was accomplished with a yield around 70%. The starting material, 4-hexylresorcinol was purchased from Aldrich.

Analytical Data

¹H-NMR (DMSO-d₆, δ): 11.25 (s, 1H, CHO); 9.7 (s, 1H); 6.35 (s, 1H); 2.55 (t, 2H, Ar—CH₂); 1.65 (bt, 2H); 1.34 (bs, 6H); 0.9 (bt, 3H)

M.P.=106-109° C. (Rif. 108-109° C.)

Description 2. [4-methylthiazol-2-yl]acetonitrile

To a solution of α-cyanothioacetamide (2 g, 20 mmol) in DMF (10 mL) and triethylamine (2.8 mL, 20 mmol), chloroacetone (1.6 mL, 20 mmol) was slowly added (dropping funnel). A sticky solid rapidly separated from the solution. The reaction mixture was heated at 40° C. for 1.5 hours. As judged by TLC (SiO₂; hexane/AcOEt 6:4, R_{f}≅0.7) the reaction was complete.

The suspension was poured onto water (110 mL) and extracted with AcOEt (3×150 mL). The combined organic phases were dried over Na₂SO4 and concentrated under vacuum.

The oily residue was chromatographed on a SiO₂ column (eluent hexane/AcOEt gradient from 8:2 to 1:1) yielding pure [4-methylthiazol-2-yl]acetonitrile (2, 1.5 g, 54%) as a light-yellow oil.

Analytical Data

¹H-NMR (DMSO-d₆, δ): 6.9 (s, 1H); 4.1 (s, 2H, CH₂—CN); 2.4 (s, 3H)

Description 3. [4-phenylthiazol-2-yl]acetonitrile

To a solution of α-cyanothioacetamide (0.5 g, 5 mmol) in DMF (3 mL) α-bromoacetophenone (1 g, 5 mmol, dissolved in 3 mL of DMF) was slowly added (dropping funnel). Approximately 30 min after the addition the mixture was heated to 70° C. for 30 min, after which the reaction was complete as judged by TLC (SiO₂; hexane/AcOEt 8:2, R_{f}≅0.3)

The dark solution was poured onto water (50 mL) and extracted with AcOEt (3×30 mL). The combined organic phases were dried over Na₂SO₄ and concentrated under vacuum.

The resultant oily residue was dissolved in EtOH (2 mL) and poured in water (30 mL). After 1 hour stirring at room temperature the formed solid was filtered off, washed with H₂O and dried under vacuum (2 h, 50° C.; 48 h, 25° C.). Pure [4-phenylthiazol-2-yl]acetonitrile (3, 0.87 g, 87%) was obtained.

Analytical Data

¹H-NMR (DMSO-d₆, δ): 7.9 (bd, 2H); 7.26-7.5 (m, 4H); 4.2 (s, 2H)

M.P.=60° C.

Description 4. [4-(4′-methoxy)phenylthiazol-2-yl]acetonitrile

A mixture of α-cyanothioacetamide (2 g, 20 mmol) and α-bromo-4-methoxyacetophenone (4.6 g, 20 mmol) in absolute EtOH (80 mL) was refluxed for 4 hours after which the reaction was complete as judged by TLC (SiO₂; hexane/AcOEt 7:3, R_{f}≅0.5). After cooling, the mixture was concentrated under vacuum to a small volume and i-PrOH was added until precipitation of a solid was apparent. After stirring overnight at room temperature the formed solid was filtered off, washed with i-PrOH and dried under vacuum (2 h, 50° C.). Pure [4-(4′-methoxy)phenylthiazol-2-yl]acetonitrile (4, 2.3 g, 50%) was obtained.

Analytical Data

¹H-NMR (DMSO-d₆, δ): 7.95 (s, 1H); 7.85 (d, 2H); 7.0 (d, 2H); 4.6 (s, 2H, CH₂—CN); 3.6 (s, 3H)

Description 5. [4-(4′-bromo)phenylthiazol-2-yl]acetonitrile

To a warm (50° C.) solution of α-cyanothioacetamide (1.08 g, 10.8 mmol) in absolute EtOH (45 mL), α-bromo-4-bromoacetophenone (3 g, 10.8 mmol) was added and the resultant mixture was refluxed for 4 hours. The reaction was complete as judged by TLC (SiO₂; hexane/AcOEt 7:3, R_{f}≅0.7). After cooling, the mixture was stirred at room temperature overnight. Then the formed solid was filtered off, washed with MeOH and dried under vacuum (2 h, 50° C.). Pure [4-(4′-bromo)phenylthiazol-2-yl]acetonitrile (5, 1.73 g, 57%), as a brownish solid, was obtained.

Analytical Data

¹H-NMR (DMSO-d₆, δ): 8.3 (s, 1H); 7.85 (d, 2H); 7.6 (d, 2H); 4.6 (s, 2H, CH₂—CN)

Description 6. [4-(4′-chloro)phenylthiazol-2-yl]acetonitrile

To a warm (50° C.) solution of α-cyanothioacetamide (1.0 g, 10 mmol) in absolute EtOH (25 mL), α-bromo-4-chloroacetophenone (2.33 g, 10 mmol) was added and the resultant mixture was refluxed for 5 hours. In TLC (SiO₂; hexane/AcOEt 7:3, R_{f}≅0.5) the reaction was complete. After cooling the mixture was stirred at room temperature for 1 hour. Then the formed solid was filtered off, washed with EtOH and dried under vacuum (2 h, 40° C.). Pure [4-(4′-chloro)phenylthiazol-2-yl]acetonitrile (6, 0.93 g, 40%), as a brownish solid, was obtained.

Analytical Data

¹H-NMR (CDCl₃+DMSO-d₆, δ): 7.9 (s+d, 3H); 7.35 (d, 2H); 4.6 (s, 2H, CH₂—CN)

M.P.=124-126° C.

Description 7. [4-(4′-phenoxy)phenylthiazol-2-yl]acetonitrile

To a warm (50° C.) solution of α-cyanothioacetamide (0.65 g, 6.52 mmol) in absolute EtOH (15 mL), a solution of α-bromo-4-chloroacetophenone (1.9 g, 6.52 mmol) in EtOH (15 mL) was added over 35′. Then the resultant mixture was refluxed for 1 hours. In TLC (SiO₂; hexane/AcOEt 7:3, R_{f}≅0.5) the reaction was complete. After cooling the mixture was concentrated under vacuum to small volume and i-PrOH was added until precipitation of a solid. After overnight stirring at room temperature the formed solid was filtered off, washed with i-PrOH and dried under vacuum (2 h, 40° C.). Pure [4-(4′-phenoxy)phenylthiazol-2-yl]acetonitrile (7.1 g, 53%) was obtained.

Analytical Data

¹H-NMR (CDCl₃+DMSO-d₆, δ): 8.1 (s, 1H); 7.95 (d, 2H); 7.4 (t, 2H); 7.2 (t, 1H); 7-7.15 (m, 4H); 4.6 (s, 2H, CH₂—CN)

Description 8. [4-(3′-methoxy)phenylthiazol-2-yl]acetonitrile

A mixture of α-cyanothioacetamide (1 g, 10 mmol) and α-bromo-3-methoxyacetophenone (2.3 g, 10 mmol) in absolute EtOH (25 mL) was refluxed for 4 hours and in TLC (SiO₂; hexane/AcOEt 7:3, R_{f}≅0.4) the reaction was complete. After cooling the mixture was concentrated under vacuum to dryness and the oily residue was chromatographed on a SiO₂ column (eluent hexane/AcOEt 8:2) to yield pure [4-(3′-methoxy)phenylthiazol-2-yl]acetonitrile (8, 0.9 g, 40%).

Analytical Data

¹H-NMR (CDCl₃, δ): 7.3-7.5 (m, 4H); 6.95 (dd, 1H); 4.2 (s, 2H, CH₂—CN); 3.9 (s, 3H, OCH₃)

Description 9. [4-(4-Imidazol-1-yl-phenyl)-thiazol-2-yl]-acetonitrile

Starting from commercially available 1-[4-(1H-imidazol-2-yl)phenyl]ethanone and according to the procedures described for compounds of formula (6) and (1) in the general synthetic schemes (2) and (1), respectively, the title compound was obtained.

(yield=54%).

Description 10. [4-(4-Nitro-phenyl)-thiazol-2-yl]-acetonitrile

Starting from commercially available 1-(4-nitrophenyl)ethanone and according to the procedures described for compounds of formula (6) and (1) in the general synthetic schemes (2) and (1), respectively, the title compound was obtained.

(yield=75%).

Description 11. 5-Ethyl-2,4-dihydroxy-benzaldehyde

According to the procedure described in Note 1 in the general synthetic schemes, the title compound was obtained as orange crystals.

(yield=63%).

Analytical Data

M.P.=124-127° C.

Description 12. 3-Acetyl-7-hydroxy-chromen-2-one

According to the procedure described in the general synthetic schemes for compounds of formula (5), in Scheme 2, the title compound was obtained starting from 2,4-dihydroxybenzaldehyde and ethylacetoacetate.

Analytical Data

M.P.=232-239° C.

Description 13. Acetic acid 3-acetyl-2-oxo-2H-chromen-7-yl ester

According to the procedure described in the general synthetic schemes for compounds of formula (8), in Scheme 2, the title compound was obtained starting from compound of Description 12.

(yield=54%).

Analytical Data

M.P.=154-156° C.

Description 14. Acetic acid 3-(2-bromo-acetyl)-2-oxo-2H-chromen-7-yl ester

According to the procedure described in the general synthetic schemes for compounds of formula (9), in Scheme 2, the title compound was obtained starting from compound of Description 13.

(yield=76%).

Analytical Data

M.P.=182-192° C.

Description 15. Acetic acid 2-oxo-3-(2-phenyl-thiazol-4-yl)-2H-chromen-7-yl ester

According to the procedure described in the general synthetic schemes for compounds of formula (10), in Scheme 2, the title compound was obtained starting from compound of Description 14 and commercially available benzenethiocarboxamide.

Analytical Data

M.P.=188-191° C.

Description 16. Acetic acid 2-oxo-3-(2-thiophen-2-yl-thiazol-4-yl)-2H-chromen-7-yl ester

According to the procedure described in the general synthetic schemes for compounds of formula (10), in Scheme 2, the title compound was obtained starting from compound of Description 14 and commercially available 2-thiophenethiocarboxamide.

Description 17. Acetic acid 2-oxo-3-(2-pyridin-3-yl-thiazol-4-yl)-2H-chromen-7-yl ester, hydrobromide

According to the procedure described in the general synthetic schemes for compounds of formula (10), in Scheme 2, the title compound was obtained starting from compound of Description 14 and 3-pyridinethiocarboxamide.

(yield=68%).

Analytical Data

M.P.=>270° C.

Description 18. Acetic acid 2-oxo-3-(2-pyridin-4-yl-thiazol-4-yl)-2H-chromen-7-yl ester, hydrobromide

According to the procedure described in the general synthetic schemes for compounds of formula (10), in Scheme 2, the title compound was obtained starting from compound of Description 14 and 4-pyridinethiocarboxamide.

(yield=68%).

Analytical Data

M.P.=>300° C.

Description 19. 3-Acetyl-6-hexyl-7-hydroxy-chromen-2-one

According to the procedure described in the general synthetic schemes for compounds of formula (5), in Scheme 2, the title compound was obtained starting from compound of Description 1 and ethylacetoacetate.

(yield=48%).

Analytical Data

M.P.=204-206° C.

Description 20. Acetic acid 3-acetyl-6-hexyl-2-oxo-2H-chromen-7-yl ester

According to the procedure described in the general synthetic schemes for compounds of formula (8), in Scheme 2, the title compound was obtained starting from compound of Description 19.

(yield=47%).

Description 21. Acetic acid 3-(2-bromo-acetyl)-6-hexyl-2-oxo-2H-chromen-7-yl ester

According to the procedure described in the general synthetic schemes for compounds of formula (9), in Scheme 2, the title compound was obtained starting from compound of Description 20.

(yield=58%).

Analytical Data

M.P.=158-160° C.

Description 22. Acetic acid 6-hexyl-2-oxo-3-(2-phenyl-thiazol-4-yl)-2H-chromen-7-yl ester

According to the procedure described in the general synthetic schemes for compounds of formula (10), in Scheme 2, the title compound was obtained starting from compound of Description 21 and benzenethiocarboxamide.

(yield=45%).

Description 23. Acetic acid 6-hexyl-2-oxo-3-(2-thiophen-2-yl-thiazol-4-yl)-2H-chromen-7-yl ester

According to the procedure described in the general synthetic schemes for compounds of formula (10), in Scheme 2, the title compound was obtained starting from compound of Description 21 and 2-thiophenethiocarboxamide.

(yield=58%).

Description 24. 7-Diethylamino-2-oxo-2H-chromene-3-carbothioic acid amide

To a solution of 4-(diethylamino)salicylaldehyde (1.93 g, 10 mmol) and α-cyanothioacetaride (1 g, 10 mmol) in absolute EtOH (100 mL) were added a few drops of piperidine. The resulting solution was then stirred for 24 hours at room temperature. A solid precipitated after 15 min. The precipitated solid was collected, washed with absolute EtOH and dried under vacuum (2.48 g, 90% yield).

Analytical Data

¹H-NMR (DMSO-d₆, δ): 11.8 (br, 1H); 9.91 (br, 1H); 8.87 (s, 1H); 7.54 (d, 1H, J=9.0 Hz); 6.63 (dd, 1H, J=2.2, 9.0); 6.32 (d, 1H, J=2.2); 3.47 (q, 4H, J=7.2); 1.12 (t, 6H, J=7.2)

Description 25. 3-(2-Bromoacetyl)-7-diethylamino-chromen-2-one, hydrobromide

To a solution of 7-diethylamino-3-acetylchromen-2-one (0.13 g, 0.5 mmol; prepared from commercially available 4-diethylaminosalicylaldehyde and ethylacetoacetate according to the procedure described for compound (5) in the general synthetic scheme (2)) in glacial acetic acid (5 mL) was added 48% HBr (113 mL, 1 mmol). Bromine was then added dropwise (26 mL, 0.5 mmol) and the resulting suspension was stirred overnight at room temperature. The solvent was evaporated under vacuum and the solid residue was triturated with Et₂O. The solid was collected and washed with Et₂O (0.148 g, 87% yield).

Analytical Data

¹H-NMR (DMSO-d₆, δ): 8.61 (s, 1H); 7.71 (d, 1H, J=9.0 Hz); 6.83 (dd, 1H, J=2.2, 9.0); 6.61 (d, 1H, J=2.2); 4.79 (s, 2H); 3.51 (q, 4H, J=7.2); 1.15 (t, 6H, J=7.2)

Description 26. 2-Bromo-1,2-diphenylethanone

According to the procedure described in the general synthetic schemes for compounds of formula (6), in Scheme 2, the title compound was obtained as a reddish oil starting from commercially available 1,2-diphenylethanone.

(yield=99%).

Analytical Data

¹H-NMR (DMSO-d₆, δ): 8.06 (m, 2H); 7.70-7.25 (m, 8H); 7.18 (s, 1H)

Specific Syntheses of Coumarins

### Example 1

### 3-[4-methylthiazol-2-yl]-6-(n-hexyl)-7-hydroxy-chromen-2-one

To a mixture of 4-methylthiazol-2-ylacetonitrile of Description 2 (2, 0.4 g, 2.89 mmol) and 5-hexyl-4-hydroxysalicyl-aldehyde of Description 1 (0.64 g, 2.89 mmol) in absolute ethanol (8 mL) piperidine (5 drops) was added and the mixture was refluxed for 1 hour. The reaction completion was checked by TLC (SiO₂; hexane/AcOEt 6:4, R_{f}≅0.2).

After cooling the precipitated solid, which corresponded to the imino derivative intermediate, was filtered off, washed with EtOH and dried under vacuum (1 h, 50° C.).

The so obtained brownish solid (0.79 g, 80% yield) was suspended in water/AcOH 1:1 (20 mL) and refluxed for almost 2 hours. The reaction was complete in TLC (hexane/AcOEt 6:4, R_{f}≅0.8).

The suspension was cooled to room temperature and the solid removed by filtration. After several washings with water the yellowish solid was dried under vacuum (2 h, 40° C.; 16 h, 25° C.) to yield pure 3-[4-methylthiazol-2-yl]-6-(n-hexyl)-7-hydroxy-chromen-2-one (0.75 g, 75% yield).

Analytical Data

¹H-NMR (DMSO-d₆, δ): 8.8 (s, 1H); 7.7 (s, 1H); 7.45 (s, 1H); 6.8 (s, 1H); 2.6 (t, 2H, CH₂—Ar); 2.4 (s, 3H); 1.5 (m, 2H); 1.2 (m, 6H); 0.85 (bt, 3H)

Elemental analysis: found % (theoretical %); C, 66.45(66.45); H, 6.14(6.16); N, 4.18(4.08); S, 9.36(9.34).

### Example 2

### 3-[4-methylthiazol-2-yl]-6-(n-hexyl)-7-acetoxy-chromen-2-one

To a cooled (0-5° C.) solution of 3-[4-methylthiazol-2-yl]-6-(n-hexyl)-7hydroxy-chromen-2-one (compound of Example 1; 0.43 g, 1.25 mmol) in anhydrous pyridine (5 mL), acetic anhydride (0.13 mL, 1.37 mmol) was added dropwise over 20 min. The cooling bath was removed and the mixture stirred for 2 hour. The reaction was complete in TLC (SiO₂; hexane/AcOEt 8:2, R_{f}≅0.8).

The precipitated solid was filtered off and washed several times with hexane. After drying under vacuum (3 h, 40° C.; overnight, 25° C.) a brownish solid was achieved (0.26 g, 54% yield).

Analytical Data

¹H-NMR (DMSO-d₆, δ): 8.9 (s, 1H); 7.9 (s, 1H); 7.4 (s, 1H); 7.3 (s, 1H); 2.3 (s, 3H, CH₃COO—Ar; CH₃-Het under DMSO peak); 1.5 (m, 2H); 1.2 (m, 6H); 0.85 (bt, 3H)

Elemental analysis: found % (theoretical %); C, 65.29(65.43); H, 5.99(6.01); N, 3.67(3.63); S, 8.22(8.32).

M.P.=136-140° C.

### Example 3

### 3-[4-phenylthiazol-2-yl]-6-(n-hexyl)-7-hydroxy-chromen-2-one

To a mixture of 4-phenylthiazol-2-yl)acetonitrile (compound of Description 3, 0.5 g, 2.49 mmol) and 5-hexyl-4-hydroxysalicyl-aldehyde (compound of Description 1, 0.55 g, 2.49 mmol) in absolute ethanol (20 mL) piperidine (8 drops) was added and the mixture was refluxed for 1 hour. The reaction completion was checked by TLC (SiO₂; hexane/AcOEt 1:1, R_{f}≅0.2).

After cooling the precipitated solid, which corresponded to the imino derivative intermediate, was filtered off, washed with i-PrOH and dried under vacuum (2 h, 50° C.).

The so obtained brownish solid (0.78 g, 77% yield) was suspended in water/AcOH 1:1 (25 mL) and refluxed for 3.5 hours. The reaction was complete in TLC (hexane/AcOEt 6:4, R_{f}≅0.8).

The suspension was cooled to room temperature and the solid removed by filtration. After several washings with water the yellowish solid was dried under vacuum (2 h, 50° C.; 72 h, 25° C.) to yield pure 3-[4-phenylthiazol-2-yl]-6-(n-hexyl)-7-hydroxy-chromen-2-one (0.73 g, 73% yield).

Analytical Data

¹H-NMR (DMSO-d₆, δ): 9.0 (s, 1H); 8.2 (s, 1H); 8.1 (d, 2H); 7.7 (s, 1H); 7.4-7.5 (m, 3H); 6.8 (s, 1H); 2.6 (t, 2H); 1.6 (m, 2H); 1.3 (m, 6H); 0.9 (bt, 3H)

Elemental analysis: found % (theoretical %); C, 69.82(71.09); H, 5.81(5.72); N, 3.46(3.45); S, 7.71(7.91).

M.P.=220-230° C.

### Example 4

### 3-[4-(4′-chloro)phenylthiazol-2-yl]-6-(n-hexyl)-7-hydroxy-chromen-2-one

To a mixture of [4-(4′-chloro)phenylthiazol-2-yl]acetonitrile (compound of Description 6, 0.23 g, 1 mmol) and 5-hexyl-4-hydroxysalicylaldehyde (compound of Description 1, 0.22 g, 1 mmol) in absolute ethanol (5 mL) piperidine (10 drops) was added and the mixture was refluxed for 40′. The reaction completion was checked by TLC (SiO₂; hexane/AcOEt 7:3, R_{f}≅0.3).

The precipitated solid, which corresponded to the imino derivative intermediate, was stirred at room temperature overnight, then it was filtered off, washed with EtOH and dried under vacuum (2 h, 50° C.).

The so obtained orange solid was suspended in water/AcOH 1:1 (15 mL) and refluxed for 4 hours. The reaction was complete in TLC (hexane/AcOEt 6:4, R_{f}≅0.8). The solid turned from orange to yellow.

The suspension was cooled to room temperature and the solid filtered off. After several washings with water the yellow solid was dried under vacuum (2 h, 50° C.) to yield pure 3-[4-(4′-chloro)phenylthiazol-2-yl]-6-(n-hexyl)-7-hydroxy-chromen-2-one (0.34 g, 77% yield).

Analytical Data

¹H-NMR (DMSO-d₆, δ): 9.0 (s, 1H); 8.25 (s, 1H); 8.1 (d, 2H); 7.7 (s, 1H); 7.55 (d, 2H); 6.8 (s, 1H); 2.6 (t, 2H); 1.6 (m, 2H); 1.3 (m, 6H); 0.9 (bt, 3H)

Elemental analysis: found % (theoretical %); C, 64.61(65.52); H, 5.08(5.04); N, 3.53(3.18); Cl, 7.94(8.06); S, 7.42(7.29).

M.P.=259-260° C.

### Example 5

### 3-[4-(4′-phenoxy)phenylthiazol-2-yl]-6-(n-hexyl)-7-hydroxy-chromen-2-one

To a mixture of [4-(4′-phenoxy)phenylthiazol-2-yl]acetonitrile (compound of Description 7, 0.5 g, 1.71 mmol) and 5-hexyl-4-hydroxysalicylaldehyde (compound of Description 1, 0.38 g, 1.71 mmol) in absolute ethanol (25 mL) piperidine (8 drops) was added and the mixture was refluxed for 1 hour. The reaction completion was checked by TLC (SiO₂; hexane/AcOEt 1:1, R_{f}≅0.2).

After cooling the precipitated solid, which corresponded to the imino derivative intermediate, was filtered off, washed with i-PrOH and dried under vacuum (1 h, 50° C.).

The so obtained brownish solid (0.63 g, 74% yield) was suspended in water/AcOH 1:1 (25 mL) and refluxed for 3 hours. The reaction was complete in TLC (SiO₂; hexane/AcOEt 6:4, R_{f}≅0.8).

The suspension was cooled to room temperature and the solid removed by filtration. After several washings with water the yellow solid was dried under vacuum (2 h, 50° C.; 72 h, 20° C.) to yield pure 3-[4-(4′phenoxy)phenylthiazol-2-yl]-6-(n-hexyl)-7-hydroxy-chromen-2-one (0.6 g, 71% yield).

Analytical Data

¹H-NMR (DMSO-d₆, δ): 9.0 (s, 1H); 8.1 (d+s, 2H+1H); 7.7 (s, 1H); 7.4 (m, 2H); 7.1 (m, 5H); 6.8 (s, 1H); 2.6 (t, 2H); 1.6 (m, 2H); 1.3 (m, 6H); 0.9 (bt, 3H)

Elemental analysis: found % (theoretical %); C, 71.66(72.41); H, 5.36(5.47); N, 2.91(2.81); S, 6.39(6.44).

M.P.=220-226° C.

### Example 6

### 3-[4-(4′-bromo)phenylthiazol-2-yl]-6-(n-hexyl)-7-hydroxy-chromen-2-one

To a mixture of [4-(4′-bromo)phenylthiazol-2-yl]acetonitrile (compound of Description 5, 0.42 g, 1.5 mmol) and 5-hexyl-4-hydroxysalicylaldehyde (compound of Description 1, 0.33 g, 1.5 mmol) in absolute ethanol (8 mL) piperidine (10 drops) was added and the mixture was refluxed for 40 min. The reaction turned rapidly to dark-red and its completion was checked by TLC (hexane/AcOEt 7:3, R_{f}≅0.3).

The mixture, which corresponded to the imino derivative intermediate, was diluted with water/AcOH 1:1 (20 mL) and refluxed for 2 hours. The reaction was complete in TLC (SiO₂; hexane/AcOEt 6:4, R_{f}≅0.8).

The suspension was cooled to room temperature and the solid filtered off. After several washings with water the yellow solid was dried under vacuum (2 h, 50° C.) to yield pure 3-[4-(4′-bromo)phenylthiazol-2-yl]-6-(n-hexyl)-7-hydroxy-chromen-2-one (0.34 g, 77% yield).

Analytical Data

¹H-NMR (DMSO-d₆, δ): 9.0 (s, 1H); 8.25 (s, 1H); 8.1 (d, 2H); 7.7 (s+d, 1H+2H); 6.8 (s, 1H); 2.6 (t, 2H); 1.6 (m, 2H); 1.3 (m, 6H); 0.9 (bt, 3H)

Elemental analysis: found % (theoretical %); C, 58.62(59.51); H, 4.72(4.58); N, 3.15(2.89); Br, 15.95(16.49); S, 6.54(6.62).

M.P.>250° C.

### Example 7

### 3-[4-methylthiazol-2-yl]-7-(N,N-diethylamino)-chromen-2-one

To a mixture of (4-methylthiazol-2-yl)acetonitrile (compound of Description 2, 0.92 g, 6.75 mmol) and 4-(N,N-diethylamino)salicylaldehyde (from Aldrich, 1.3 g, 6.7 mmol) in absolute ethanol (25 mL) piperidine (8 drops) was added and the mixture was refluxed for 40′. The reaction turned rapidly to dark-red and its completion was checked by TLC (SiO₂; hexane/AcOEt 6:4, R_{f}≅0.2).

The mixture, which corresponded to the imino derivative intermediate, was concentrated under vacuum to a dark-brown oily residue. The sticky material was diluted with water/AcOH 1:1 (30 mL) and refluxed for 3 hours. The reaction was complete in TLC (SiO₂; hexane/AcOEt 6:4, R_{f}≅0.8).

The suspension was cooled to room temperature and stirred overnight. The precipitated solid was filtered off and washed several times with water. After drying under vacuum (3 h, 50° C.) pure 3-[4-methylthiazol-2-yl]-7-(N,N-diethylamino)-chromen-2-one (1.9 g, 90% yield) was achieved.

Analytical Data

¹H-NMR (DMSO-d₆, δ): 8.75 (s, 1H); 7.7 (d, 1H); 6.8 (dd, 1H); 6.6 (s, 1H); 3.5 (q, 4H); 2.35 (s, 3H); 1.15 (t, 6H)

Elemental analysis: found % (theoretical %); C, 64.82(64.94); H, 5.78(5.77); N, 8.96(8.91); S, 9.88(10.20).

M.P.=165-166° C.

### Example 8

### 3-[4-phenylthiazol-2-yl]-7-(N,N-diethylamino)-chromen-2-one

To a mixture of (4-phenylthiazol-2-yl)acetonitrile (compound of Description 3, 1.6 g, 7.98 mmol) and 4-(N,N-diethylamino)salicylaldehyde (from Aldrich, 1.54 g, 7.98 mmol) in absolute ethanol (30 mL) piperidine (8 drops) was added and the mixture was refluxed for 40′. The reaction turned rapidly to dark and its completion was checked by TLC (SiO₂; hexane/AcOEt 6:4, R_{f}≅0.2).

The mixture, which corresponded to the imino derivative intermediate, was concentrated under vacuum to a dark-brown oily residue. The sticky material was diluted with water/AcOH 1:1 (50 mL) and refluxed for 4 hours. The reaction was complete in TLC (SiO₂; hexane/AcOEt 6:4, R_{f}≅0.8).

The suspension was cooled to room temperature and the precipitated solid was filtered off and washed several times with water. After drying under vacuum (3 h, 50° C.; 16 h, 25° C.) pure 3-[4-phenylthiazol-2-yl]-7-(N,N-diethylamino)-chromen-2-one (2.8 g, 93% yield) was achieved.

Analytical Data

¹H-NMR (DMSO-d₆, δ): 8.9 (s, 1H); 8.1 (s+d, 1H+2H); 7.75 (d, 1H); 7.4 (m, 3H); 6.8 (d, 1H); 6.65 (s, 1H); 3.5 (q, 4H); 1.15 (t, 6H)

Elemental analysis: found % (theoretical %); C, 70.14(70.19); H, 5.33(5.35); N, 7.52(7.44); S, 8.73(8.52).

M.P.=125-126° C.

### Example 9

### 3-[4-phenylthiazol-2-yl]-7-hydroxy-chromen-2-one

To a mixture of (4-phenylthiazol-2-yl)acetonitrile (compound of Description 3, 2.5 g, 12.5 mmol) and 2,4-dihydroxybenzaldehyde (from Aldrich, 1.72 g, 12.5 mmol) in absolute ethanol (30 mL) piperidine (8 drops) was added and the mixture was refluxed for 4 hour. The reaction completion was checked by TLC (SiO₂; hexane/AcOEt 1:1, R_{f}≅0.3).

The mixture was stirred overnight at room temperature. Then the precipitated solid, which corresponded to the imino derivative intermediate, was filtered off, washed with EtOH and dried under vacuum (2 h, 50° C.). The resultant crude material was chromatographed on SiO₂ column (eluent hexane/AcOEt 6:4) to yield, after crystallization from EtOH/AcOEt 1:1, 2.1 g of pure imino compound which was characterised by ¹H-NMR [DMSO-d₆, δ: 8.8 (s, 1H); 8.6 (s, 1H, ═N—H); 8.15 (s, 1H, Thiaz-H, 8.1 (d, 2H); 7.6 (d, 1H); 7.5 (t, 2H); 7.4 (d, 1H); 6.7 (dd, 1H); 6.6 (d, 1H)].

The so obtained solid (2.1 g, 52% yield) was suspended in water/AcOH 1:1 (25 mL) and refluxed for 2 hours. The reaction was complete in TLC (SiO₂; hexane/AcOEt 6:4, R_{f}≅0.8).

The suspension was cooled to room temperature and the solid removed by filtration. After several washings with water the resultant solid was dried under vacuum (4 h, 40° C.) to yield pure 3-[4-phenylthiazol-2-yl]-7-hydroxy-chromen-2-one (1.98 g, 49% yield).

Analytical Data

¹H-NMR (DMSO-d₆, δ): 9.1 (s, 1H); 8.2 (s, 1H, Thiaz-H); 8.1 (d, 2H); 7.9 (d, 1H); 7.3-7.55 (m, 3H); 6.9 (dd, 1H); 6.85 (d, 1H)

Elemental analysis: found % (theoretical %); C, 67.25(67.28); H, 3.42(3.45); N, 4.38(4.36); S, 10.06(9.98).

M.P.=>300° C.

### Example 10

### 3-[4-phenylthiazol-2-yl]-7-acetoxy-chromen-2-one

To a cooled (0-5° C.) solution of 3-[4-phenylthiazol-2-yl]-7-hydroxy-chromen-2-one (compound of Example 9; 0.5 g, 1.37 mmol) in anhydrous pyridine (5 mL), acetic anhydride (0.143 mL, 1.51 mmol) was added dropwise over 20 min. The cooling bath was removed and the mixture stirred overnight. The reaction was complete in TLC (SiO₂; hexane/AcOEt 1:1, R_{f}≅0.8).

The precipitated solid was filtered off and washed several times with hexane. After drying under vacuum (5 h, 40° C.; overnight, 25° C.) a brownish solid was achieved (0.32 g, 64% yield).

Analytical Data

¹H-NMR (DMSO-d₆, 5): 9.15 (s, 1H); 8.35 (s, 1H); 8.1 (m, 3H); 7.35-7.55 (m, 4H); 7.25 (dd, 1H); 2.35 (s, 3H)

Elemental analysis: found % (theoretical %); C, 66.33(66.10); H, 3.61(3.61); N, 3.88(3.88); S, 8.80(8.82).

### Example 11

### 3-[4-(3′-methoxy)phenylthiazol-2-yl]-7-hydroxy-chromen-2-one

To a mixture of [4-(3′-methoxy)phenylthiazol-2-yl]acetonitrile (compound of Description 8, 0.45 g, 1.95 mmol) and 2,4-dihydroxybenzaldehyde (from Aldrich, 0.27 g, 1.95 mmol) in absolute ethanol (5 mL) piperidine (5 drops) was added and the mixture was refluxed for 5 hour. The reaction completion was checked by TLC (SiO₂; hexane/AcOEt 1:1, R_{f}≅0.3).

The precipitated solid, which corresponded to the imino derivative intermediate, was filtered off, washed with EtOH and dried under vacuum (2 h, 30° C.) to yield 0.655 g of fluorescent yellow solid.

The so obtained material (0.635 g, 93% yield) was suspended in water/AcOH 1:1 (25 mL) and refluxed for 2 hours. The reaction was complete in TLC (SiO₂; hexane/AcOEt 6:4, R_{f}≅0.9).

The suspension was cooled to room temperature and the solid removed by filtration. After several washings with water the resultant solid was dried under vacuum (2 h, 60° C.; 24 h, 25° C.) to yield pure 3-[4-(3′-methoxy)phenylthiazol-2-yl]-7-hydroxy-chromen-2-one (0.6 g, 94% yield).

Analytical Data

¹H-NMR (DMSO-d₆, δ): 9.1 (s, 1H); 8.2 (s, 1H); 8.1 (d, 2H); 7.9 (d, 1H); 7.3-7.55 (m, 3H); 6.9 (dd, 1H); 6.85 (d, 1H); 3.85 (s, 3H, OCH₃)

Elemental analysis: found % (theoretical %); C, 64.56(64.95); H, 3.72(3.73); N, 3.99(3.99); S, 8.74(9.13).

M.P.=275-276° C.

### Example 12

### 3-[4-(4′-methoxy)phenylthiazol-2-yl]-7-hydroxy-chromen-2-one

To a mixture of [4-(4′-methoxy)phenylthiazol-2-yl]acetonitrile (compound of Description 4, 0.31 g, 1.34 mmol) and 2,4-dihydroxybenzaldehyde (from Aldrich, 0.186 g, 1.34 mmol) in absolute ethanol (5 mL) piperidine (5 drops) was added and the mixture stirred overnight at room temperature. The reaction completion was checked by TLC (SiO₂; CH₂Cl₂/AcOEt 1:1, R_{f}≅0.2).

The precipitated solid, which corresponded to the imino derivative intermediate, was filtered off, washed with EtOH and dried under vacuum (2 h, 30° C.) to yield 0.42 g of a yellow solid.

The so obtained material (0.385 g, 81% yield) was suspended in water/AcOH 1:1 (10 mL) and refluxed for 1.45 hours. The reaction was complete in TLC (SiO₂; hexane/AcOEt 6:4, R_{f}≅0.9).

The orange suspension was cooled to room temperature and the solid removed by filtration. After several washings with water the resultant solid was dried under vacuum (2 h, 60° C.; 24 h, 25° C.) to yield pure 3-[4-(4′-methoxy)phenylthiazol-2-yl]-7-hydroxy-chromen-2-one (0.38 g, 98% yield).

Analytical Data

¹H-NMR (DMSO-d₆, δ): 9.05 (s, 1H); 8.05 (s+d, 1H+2H); 7.9 (d, 1H); 7.05 (d, 2H); 6.9 (dd, 1H); 6.85 (d, 1H); 3.8 (s, 3H, OCH₃)

Elemental analysis: found % (theoretical %); C, 64.95(63.76); H, 3.73(3.63); N, 3.99(4.01); S, 9.13(8.87).

M.P.=300° C.

### Example 13

### 3-[4-(4′-phenoxy)phenylthiazol-2-yl]-7-hydroxy-chromen-2-one

To a mixture of [4-(4′-phenoxy)phenylthiazol-2-yl]acetonitrile (compound of Description 7, 1.2 g, 4.1 mmol) and 2,4-dihydroxybenzaldehyde (from Aldrich, 0.56 g, 4.1 mmol) in absolute ethanol (15 mL) piperidine (5 drops) was added and the mixture stirred overnight at room temperature. The reaction completion was checked by TLC (SiO₂; CH₂Cl₂/AcOEt 6:4, R_{f}≅0.2).

The precipitated solid, which corresponded to the imino derivative intermediate; was filtered off, washed with EtOH and dried under vacuum (2 h, 30° C.) to yield 0.9 g of an orange solid.

The so obtained material (0.88 g, 52% yield) was suspended in water/AcOH 1:1 (35 mL) and refluxed for 3 hours. The reaction was complete in TLC (SiO₂; hexane/AcOEt 6:4, R_{f}≅0.9).

The yellow suspension was cooled to room temperature and the solid removed by filtration. After several washings with water the yellow solid was dried under vacuum (3 h, 60° C.; 48 h, 25° C.) to yield pure 3-[4-(4′-phenoxy)phenylthiazol-2-yl]-7-hydroxy-chromen-2-one (0.71 g, 81% yield).

Analytical Data

¹H-NMR (DMSO-d₆, δ): 9.05 (s, 1H); 8.15 (d, 2H); 8.1 (s, 1H); 7.85 (d, 1H); 7.4 (dd, 2H); 7.0-7.2 (m, 5H); 6.9 (dd, 1H); 6.8 (d, 1H)

Elemental analysis: found % (theoretical %); C, 68.99(69.72); H, 3.67(3.66); N, 3.54(3.39); S, 7.64(7.76).

### Example 14

### 3-[4-(4′-bromo)phenylthiazol-2-yl]-7-hydroxy-chromen-2-one

To a mixture of 4-(4′-bromo)phenylthiazol-2-ylacetonitrile (compound of Description 5, 1.7 g, 6.09 mmol) and 2,4-dihydroxybenzaldehyde (from Aldrich, 0.84 g, 6.09 mmol) in absolute ethanol (25 mL) piperidine (0.17 mL) was added and the mixture refluxed for 4 hours. The reaction completion was checked by TLC (SiO₂; CH₂Cl₂/AcOEt 6:4, R_{f}≅0.2).

The precipitated solid, which corresponded to the imino derivative intermediate, was filtered off, washed with EtOH and dried under vacuum (2 h, 30° C.) to yield 1.9 g of a brownish solid.

The so obtained material (1.9 g, 49% yield) was suspended in water/AcOH 1:1 (20 mL) and refluxed for 4 hours. The reaction was complete in TLC (SiO₂; hexane/AcOEt 1:1, R_{f}≅0.9).

The suspension was cooled to room temperature and the solid removed by filtration. After several washings with water the solid was dried under vacuum (3 h, 40° C.) to yield pure 3-[4-(4′-bromo)phenylthiazol-2-yl]-7-hydroxy-chromen-2-one (1.8 g, 94% yield).

Analytical Data

¹H-NMR (DMSO-d₆, 5): 9.05 (s, 1H); 8.15 (d, 2H); 8.1 (s, 1H); 7.9 (d, 2H); 7.85 (d, 1H); 7.4 (dd, 2H); 7.0-7.2 (m, 5H); 6.9 (dd, 1H); 6.8 (d, 1H)

Elemental analysis: found % (theoretical %); C, 53.92(54.02); H, 2.53(2.52); N, 3.87(3.50); S, 8.06(8.01); Br, 19.69(19.96).

M.P.=>300° C.

### Example 15

### 6-Hexyl-7-hydroxy-3-[4-(4-imidazol-1-yl-phenyl)-thiazol-2-yl]-chromen-2-one

Starting from the compounds of Description 1 and 9 and following the procedure described for compounds of formula (2), method A, in the general synthetic schemes, the title compound was obtained.

(yield=61%).

Analytical Data

¹H-NMR (DMSO-d₆, δ): 10.9 (br, 1H); 9.06 (s, 1H); 8.37 (br s, 1H); 8.30 (s, 1H); 8.23 (d, 2H); 7.85 (br s, 1H); 7.78 (d, 2H); 7.76 (s, 1H); 7.15 (br s, 1H); 6.86 (s, 1H); 2.59 (t, 2H); 1.60 (quintet, 2H); 1.30 (m, 6H); 0.87 (t, 3H)

Elemental analysis: found % (theoretical %); C, 67.66(68.77); H, 5.39(5.34); N, 8.79(8.91); S, 6.90(6.80).

M.P.=245-247° C.

### Example 16

### 6-Hexyl-7-hydroxy-3-[4-(4-nitro-phenyl)-thiazol-2-yl]-chromen-2-one

Starting from the compounds of Description 1 and 10 and following the procedure described for compounds of formula (2), method A, in the general synthetic schemes, the title compound was obtained.

(yield=49%).

Analytical Data

¹H-NMR (DMSO-d₆, δ): 10.98 (br, 1H); 9.00 (s, 1H); 8.51 (s, 1H); 8.31 (m, 4H); 7.70 (s, 1H); 6.82 (s, 1H); 2.56 (t, 2H); 1.58 (quintet, 2H); 1.30 (m, 6H); 0.87 (t, 3H)

Elemental analysis: found % (theoretical %); C, 64.06(63.99); H, 4.79(4.92); N, 6.20(6.22); S, 7.16(7.12).

M.P.=>280° C.

### Example 17

### 6-Hexyl-7-hydroxy-3-[4-(3-nitro-phenyl)-thiazol-2-yl]-chromen-2-one

Starting from the compound of Description 1 and from 1-(3-nitrophenyl)-2-bromoethanone (obtained in the same way as the compound of Description 10 starting from commercially available 1-(3-nitrophenyl)ethanone and bromine) following the procedure described for compounds of formula (2), method B, in the general synthetic schemes, the title compound was obtained.

(yield=53%).

Analytical Data

¹H-NMR (DMSO-d₆, 5): 11.0 (br, 1H); 9.05 (s, 1H); 8.88 (t, 1H); 8.55 (br d, 1H); 8.51 (s, 1H); 8.24 (dd, 1H); 7.79 (t, 1H); 7.79 (s, 1H); 6.85 (s, 1H); 2.59 (t, 2H); 1.58 (quintet, 2H); 1.30 (m, 6H); 0.87 (t, 3H)

Elemental analysis: found % (theoretical %); C, 63.68(63.99); H, 4.82(4.92); N, 6.23(6.22); S, 7.21(7.12).

M.P.=241-242° C.

### Example 18

### 6-Hexyl-7-hydroxy-3-[4-(3-methoxy-phenyl)-thiazol-2-yl]-chromen-2-one

Starting from the compounds of Description 1 and 8 and following the procedure described for compounds of formula (2), method A, in the general synthetic schemes, the title compound was obtained.

(yield=33%).

Analytical Data

¹H-NMR (DMSO-d₆, δ): 10.94 (br, 1H); 9.04 (s, 1H); 8.24 (s, 1H); 7.76 (m, 2H); 7.40 (t, 1H); 6.96 (ddd, 1H); 6.86 (s, 1H); 3.86 (s.3H); 2.59 (t, 2H); 1.58 (quintet, 2H); 1.30 (m, 6H); 0.87 (t, 3H)

Elemental analysis: found % (theoretical %); C, 67.95(68.94); H, 5.74(5.79); N, 3.36(3.22); S, 7.24(7.36).

M.P.=198-200° C.

### Example 19

### 6-Hexyl-7-hydroxy-3-[4-(4-methoxy-phenyl)-thiazol-2-yl]-chromen-2-one

Starting from the compounds of Description 1 and 4 and following the procedure described for compounds of formula (2), method A, in the general synthetic schemes, the title compound was obtained.

(yield=62%).

Analytical Data

¹H-NMR (DMSO-d₆, δ): 10.95 (br, 1H); 9.00 (s, 1H); 8.04 (s, 1H); 8.02 (d, 2H); 7.72 (s, 1H); 7.05 (d, 2H); 6.84 (s, 1H); 3.81 (s, 3H); 2.59 (t, 2H); 1.58 (quintet, 2H); 1.30 (m, 6H); 0.87 (t, 3H)

Elemental analysis: found % (theoretical %); C, 68.84(68.94); H, 5.70(5.79); N, 3.31(3.22); S, 7.45(7.36).

M.P.=239-240° C.

### Example 20

### 6-Ethyl-7-hydroxy-3-(4-phenyl-thiazol-2-yl)-chromen-2-one

Starting from the compounds of Description 11 and 3 and following the procedure described for compounds of formula (2), method A, in the general synthetic schemes, the title compound was obtained.

Analytical Data

¹H-NMR (DMSO-d₆, δ): 11.0 (br, 1H); 9.04 (s, 1H); 8.21 (s, 1H); 8.11 (d, 2H); 7.77 (s, 1H); 7.60-7.35 (m, 3H); 6.86 (s, 1H); 2.62 (q, 2H); 1.21 (t, 3H)

Elemental analysis: found % (theoretical %); C, 68.47(68.47); H, 4.41(4.33); N, 4.10(4.01); S, 9.07(9.17).

M.P.=289-292° C.

### Example 21

### 6-Nitro-3-(4-phenyl-thiazol-2-yl)-chromen-2-one

Starting from the compounds of Description 3 and the commercially available 5-nitrosalicylaldehyde and following the procedure described for compounds of formula (2), method A, in the general synthetic schemes, the title compound was obtained.

(yield=57%).

Analytical Data

¹H-NMR (DMSO-d₆, δ): 9.32 (s, 1H); 9.05 (d, 1H); 8.48 (dd, 1H); 8.38 (s, 1H); 8.12 (m, 2H); 7.74 (d, 1H); 7.60-7.35 (m, 3H)

Elemental analysis: found % (theoretical %); C, 61.61(61.71); H, 3.03(2.88); N, 8.05(8.00); S, 9.10(9.15).

M.P.=>270° C.

### Example 22

### {3-[4-(4-Bromo-phenyl)-thiazol-2-yl]-2-oxo-2H-chromen-7-yloxy}-acetic acid ethyl ester

Starting from the compound of Example 14 and following the procedure described for compounds of formula (4), in the general synthetic schemes, the title compound was obtained.

(yield=56%).

Analytical Data

¹H-NMR (DMSO-d₆, δ): 9.08 (s, 1H); 8.33 (s, 1H); 8.06 (d, 2H); 7.98 (d, 1H); 7.69 (d, 21); 7.16 (d, 1H); 7.10 (dd, 1H); 5.00 (s, 2H); 4.20 (q, 2H); 1.24 (t, 3H)

Elemental analysis: found % (theoretical %); C, 54.25(54.33); H, 3.39(3.32); N, 2.88(2.88); Br, 16.18(16.43); S, 6.58(6.59).

M.P.=258-260° C.

### Example 23

### 7-Hydroxy-3-(2-phenyl-thiazol-4-yl)-chromen-2-one

Starting from the compound of Description 15 and following the procedure described for compounds of formula (11), in the general synthetic schemes, the title compound was obtained.

(yield=70%).

Analytical Data

¹H-NMR (DMSO-d₆, δ): 10.70 (br, 1H); 8.90 (s, 1H); 8.33 (s, 1H); 8.07 (m, 2H); 7.78 (d, 1H); 7.55 (m, 3H); 6.88 (dd, 1H); 6.81 (d, 1H)

Elemental analysis: found % (theoretical %); C, 65.82 (67.28); H, 3.48(3.45); N, 4.23(4.36); S, 9.65(9.97).

M.P.=270-275° C.

### Example 24

### 7-Hydroxy-3-(2-(thiophen-2-yl)-thiazol-4-yl)-chromen-2-one

Starting from the compound of Description 16 and following the procedure described for compounds of formula (11), in the general synthetic schemes, the title compound was obtained.

(yield=79%).

Analytical Data

¹H-NMR (DMSO-d₆, δ): 10.72 (br, 1H); 8.72 (s, 1H); 8.28 (s, 1H); 7.85-7.70 (m, 3H); 7.21 (dd, 1H); 6.85 (dd, 1H); 6.80 (d, 1H)

Elemental analysis: found % (theoretical %); C, 57.91(58.70); H, 2.85(2.77); N, 4.10(4.28); S, 19.07(19.58).

M.P.=>290° C.

### Example 25

### 7-Hydroxy-3-(2-(pyridin-3-yl)-thiazol-4-yl)-chromen-2-one

Starting from the compounds of Description 17 and following the procedure described for compounds of formula (11) (note 4), in the general synthetic schemes, the title compound was obtained.

(yield=34%).

Analytical Data

Elemental analysis: found % (theoretical %); C, 62.20(63.34); H, 3.22(3.13); N, 8.52(8.69); S, 9.38(9.94).

M.P.=>250° C.

### Example 26

### 7-Hydroxy-3-(2-(pyridin-4-yl)-thiazol-4-yl)-chromen-2-one

Starting from the compounds of Description 18 and following the procedure described for compounds of formula (11) (note 4), in the general synthetic schemes, the title compound was obtained.

(yield=69%).

Analytical Data

¹H-NMR (DMSO-d₆, δ): 10.90 (br, 1H); 8.97 (d, 2H); 8.92 (s, 1H); 8.63 (s, 1H); 8.45 (d, 2H); 7.74 (d, 1H); 6.90 (dd, 1H); 6.81 (d, 1H)

Elemental analysis: found % (theoretical %); C, 58.78(63.34); H, 3.13(3.13); N, 7.92(8.69); S, 8.83(9.95).

### Example 27

### 6-Hexyl-7-hydroxy-3-(2-phenyl-thiazol-4-yl)-chromen-2-one

Starting from the compounds of Description 22 and following the procedure described for compounds of formula (11), in the general synthetic schemes, the title compound was obtained.

(yield=82%).

Analytical Data

¹H-NMR (DMSO-d₆, δ): 10.70 (br, 1H); 8.88 (s, 1H); 8.32 (s, 1H); 8.07 (m, 2H); 7.64 (s, 1H); 7.55 (m, 3H); 6.82 (s, 1H); 2.58 (t, 2H); 1.58 (quintet, 2H); 1.30 (m, 6H); 0.87 (t, 3H)

Elemental analysis: found % (theoretical %); C, 69.90(71.09); H, 5.67(5.72); N, 3.53(3.45); S, 7.55(7.90).

### Example 28

### 6-Hydroxy-3-(4-phenyl-thiazol-2-yl)-chromen-2-one

Starting from the compounds of Description 3 and the commercially available 5-hydroxysalicylaldehyde following the procedure described for compounds of formula (2), method A, in the general synthetic schemes, the title compound was obtained.

(yield=8%).

Analytical Data

¹H-NMR (DMSO-d₆, δ): 9.94 (br, 1H); 9.07 (s, 1H); 8.30 (s, 1H); 8.11 (m, 2H); 7.6-7.3 (m, 5H); 7.13 (dd, 1H)

Elemental analysis: found % (theoretical %); C, 65.47(67.28); H, 3.88(3.45); N, 4.01(4.36).

M.P.=242-295° C.

### Example 29

### 2-[4-(7-Diethylamino-2-oxo-2H-chromen-3-yl)-thiazol-2-yl]-acetonitrile

A suspension of 3-(2-Bromoacetyl)-7-diethylamino-chromen-2-one hydrobromide (compound of Description 25, 0.419 g, 1 mmol), α-cyanothioacetamide (from Aldrich, 0.1 g, 1 mmol) and sodium hydrogencarbonate (0.168 g, 2 mmol) in absolute ethanol (10 mL) was refluxed for 3 hours. More α-cyanothioacetamide (0.02 g, 0.2 mmol) was added and the resulting mixture was refluxed for 2 hours. The solvent was removed under vacuum. The solid residue was suspended in CH₂Cl₂ and the insoluble material was filtered off. The filtrate was purified by column chromatography on a silica gel (eluent CH₂Cl₂) to yield, after trituration with Et₂O, 0.22 g (65% yield) of pure product.

Analytical Data

¹H-NMR (DMSO-d₆, δ): 8.59 (s, 1H); 8.19 (s, 1H); 7.64 (d, 1H, J=9.0 Hz); 6.74 (dd, 1H, J=2.2, 9.0); 6.58 (d, 1H, J=2.2); 4.61 (s, 2H); 3.45 (q, 4H, J=7.0); 1.13 (t, 6H, J=7.0)

Elemental analysis: found % (theoretical %); C, 62.99(63.70); H, 4.96(5.05); N, 12.15(12.38); S, 9.27(9.45).

M.P.=178-179° C.

### Example 30

### 2-[4-(7-Diethylamino-2-oxo-2H-chromen-3-yl)-thiazol-2-yl]-acetic acid ethyl ester

To a cooled (0-5° C.) suspension of 2-[4-(7-Diethylamino-2-oxo-2H-chromen-3-yl)-thiazol-2-yl]-acetonitrile (compound of Example 29, 0.3 g, 0.9 mmol) in absolute ethanol (10 mL) was added 98% sulphuric acid (4.3 mL). The resulting solution was refluxed for 5.5 hours. After cooling, the solution was diluted with water and made alkaline (pH 8-9) with solid Na₂CO₃. The precipitated solid was collected, washed with water and was purified by column chromatography on silica gel (eluting mixture CH₂Cl₂/AcOEt 98:2). The solid recovered from the column was recromatographated on silica gel (eluting mixture petroleum ether/AcOEt 7:3) to yield 0.155 g (45% yield) of pure product.

Analytical Data

¹H-NMR (DMSO-d₆, δ): 8.59 (s, 1H); 8.14 (s, 1H); 7.63 (d, 1H, J=9.0 Hz); 6.74 (dd, 1H, J=2.2, 9.0); 6.58 (d, 1H, J=2.2); 4.21 (s, 2H); 4.16 (q, 2H, J=7.1); 3.45 (q, 4H, J=7.0); 1.22 (t, 3H, J=7.1); 1.13 (t, 6H, J=7.0)

M.P.=88-90° C.

### Example 31

### 2-[4-(7-Diethylamino-2-oxo-2H-chromen-3-yl)-thiazol-2-yl]-acetamide

A solution of 2-[4-(7-Diethylamino-2-oxo-2H-chromen-3-yl)-thiazol-2-yl]-acetonitrile (compound of Example 29, 0.2 g, 0.6 mmol) in 37% HCl (4 mL) was stirred for 24 hours at 17° C. The acidic solution was made alkaline (pH 11) with 1M NaOH and 12.5 N NaOH. The precipitated solid was collected, washed with water and crystallized from AcOEt to yield 0.069 g (33% yield) of pure compound.

Analytical Data

¹H-NMR (DMSO-d₆, δ): 8.59 (s, 1H); 8.08 (s, 1H); 7.70 (br, 1H); 7.61 (d, 1H, J=9.0 Hz); 7.18 (br, 1H); 6.74 (dd, 1H, J=2.2, 9.0); 6.58 (d, 1H, J=2.2); 3.92 (s, 2H); 3.45 (q, 4H, J=7.0); 1.13 (t, 6H, J=7.0)

Elemental analysis: found % (theoretical %); C, 60.14(60.49); H, 5.33(5.36); N, 11.45(11.76); S, 8.74(8.97).

M.P.=235-236° C.

### Example 32

### 2-[4-(7-Diethylamino-2-oxo-2H-chromen-3-yl)-thiazol-2-yl]-acetic acid, sodium salt

A solution of 2-[4-(7-Diethylamino-2-oxo-2H-chromen-3-yl)-thiazol-2-yl]-acetonitrile (compound of Example 29, 0.2 g, 0.6 mmol) in 37% HCl (4 mL) was stirred for 24 hours at 17° C. The acidic solution was made alkaline (pH 11) with 1M NaOH and 12.5 N NaOH. The precipitated solid was collected, washed with water and suspended in boiling AcOEt to yield 0.055 g (24% yield) of pure compound.

Analytical Data

¹H-NMR (DMSO-d₆, δ): 8.59 (s, 1H); 7.96 (s, 1H); 7.61 (d, 1H, J=9.0 Hz); 6.73 (dd, 1H, J=2.2, 9.0); 6.56 (d, 1H, J=2.2); 3.61 (s, 2H); 3.44 (q, 4H, J=7.0); 1.13 (t, 6H, J=7.0) M.P.>250° C.

### Example 33

### 7-Diethylamino-3-(4,5-diphenylthiazol-2-yl)-chromen-2-one

A suspension of 7-diethylamino-2-oxo-2H-chromene-3-carbothioic acid amide (compound of Description 24, 0.608 g, 2.2 mmol) and 2-bromo-1,2-diphenylethanone (compound of Description 26, 0.63 g, 2.2 mmol) in ethanol (25 mL) was refluxed for 5.5 hours. After cooling, the solid was collected and dissolved in CH₂Cl₂. The resulting solution was filtered on a silica gel column to yield, after trituration with Prⁱ₂O and ethanol, 0.383 g (38% yield) of pure compound.

Analytical Data

¹H-NMR (DMSO-d₆, δ): 8.87 (s, 1H); 7.76 (d, 1H, J=9.0 Hz); 7.53 (m, 2H); 7.35 (m, 8H); 6.83 (dd, 1H, J=2.2, 9.0); 6.67 (d, 1H, J=2.2); 3.49 (q, 4H, J=7.0); 1.15 (t, 6H, J=7.0)

Elemental analysis: found % (theoretical %); C, 73.83(74.31); H, 5.45(5.35); N, 6.15(6.19); S, 6.83(7.084).

M.P.>250° C.

### Example 34

### 7-Diethylamino-3-(2-phenyl-thiazol-4-yl)-chromen-2-one hydrochloride

Starting from 3-(2-bromoacetyl)-7-diethylamino-chromen-2-one, hydrobromide (compounds of description 25) and benzethiocarboxamide and according to the procedures described for compounds (7) in the general scheme (2), the title compound has been obtained. Yield 62%

Analytical data

Elemental analysis: found % (theoretical %); C, 61.14(63.99); H, 4.99(5.13); Cl, 8.22(8.59); N, 6.46(6.78); S, 7.32(7.76).

M.P.=>189-194° C.

Cell-Based Assay of VEGF Gene Transcription (VEGF-Luciferase Assay).

The activity of compounds is determined by a cell-based reporter assay which uses the hepatoma 3B (Hep3B) cell line.

This assay involves the use of a luciferase reporter gene under the direct control of the VEGF promoter. Induction of the hypoxic response using desferoxamine leads to the transcription of luciferase through activation of the VEGF promoter, which in turn leads to an increase in luciferse activity, which can be measured using most of commercially available luciferase assay kits. Molecules which inhibit the activation of the VEGF promoter can thus be detected.

This assay can be run using a Hep3B cell line which stably expresses the VEGF-luciferase construct.

Hep3B cells (ATCC Ref. No. HB-8064) are plated in 6-well plates at 2.5×10⁵ cells/well in 2 mL DMEM/10% FCS and are transfected the following day using Fugene 6 (Roche Biochemicals®). Transfection mixtures per well contain 6 μL Fugene 6 transfection reagent, 1 μg of pxp2-VEGF-luciferase reporter (rat VEGF promoter, NCBI GenBank accession no. U22373, Levy et al., 1995), plus pcDNA3.1(+) Neomycin resistance vector (INVITROGEN). Transfection is performed as recommended by manufacturer.

Cloning is performed in order to select the appropriate cell population.

The test is run with selected stable transfected cells.

For the inhibition of the VEGF gene transcription, the cells are plated at day 1 (1×10⁴ cells/well in 100 μl DMEM/10% FCS) and compounds are added the following day dissolved in 100% DMSO and diluted with DMEM/10% FCS to achieve a final highest DMSO concentration of 0.5%. After 1 hour incubation, at 37° C., desferoxamine mesylate (SIGMA) 100 μM in DMEM/10% FCS is added and the incubation time is extended for 18 hours.

Luciferase activity is measured using the Bright Glo Luciferase Assay System (Promega®, see also technical Manual, Part #TM052, Instructions for Use of Products E2620 and E2650, revised 10/00). IC50 data (concentration of compound required to cause a 50% reduction of the luciferase signal), for several compounds of the present invention are determined using this assay.

Cell-Based Assay of VEGF Production (VEGF-ELISA)

The above described HEP-3B cell line which stably expresses the VEGF-luciferase construct is used in this assay, employing the quantitative sandwich enzyme immunoassay technique.

A monoclonal antibody (R&D Systems®, Abingdon, Oxon, UK) specific for VEGF was pre-coated onto a microplate. To this was added a sample containing VEGF. After washing, a second anti-VEGF antibody coupled to horseradish peroxidase was added. After incubation and washing, the amount of bound antibody, and hence VEGF, was measured using a colorigenic substrate for horseradish peroxidase. Typically, VEGF-transfected Hep3B cells were plated at a concentration of 1.0×10⁴ cells/well in the same conditions as specified for the VEGF-Luciferase assay. Cells are then treated with the compounds as in the above assay and incubated with 100 μM desferoxamine for 17 hours at 37° C. 200 μL of supernatant were removed and the VEGF quantitated using the Quantikine® ELISA kit from R&D Systems® (catalog # DVE00) exactly according to the manufacturer's instructions. The assay is calibrated each time using recombinant human VEGF.

IC50 data (concentration of compound required to cause a 50% inhibition of the absorbance signal; or a different % inhibition, if indicated), for several compounds of the present invention, are determined using this assay.

Cytotoxicity Assay

The Hep3B cell line (ATCC Ref. No. HB-8064) is used. Cells are plated in a 96-well plate at 1×10⁴ cells/well in the same conditions used in the VEGF-Luciferase assay. Different concentrations of compounds and 100 μM desferoxamine dissolved as in the VEGF-Luciferase assay are added the following day and cells are incubated for 18 hours. Then cell proliferation is assessed using the Cell Proliferation Reagent WST-1 (Cat. No. 1 644 807) from Roche Molecular Biochemicals, according to the supplier's protocol. Briefly, the Cell Proliferation Reagent WST-1 is a colorimetric assay for the quantitation of cell proliferation and cell viability, based on the cleavage of the tetrazolium salt WST-1 by mitochondrial dehydrogenases in viable cells. Whether or not a particular compound exhibited toxicity at a particular concentration is determined using this assay.

IC50 data (concentration of compound required to cause a 50% inhibition of the proliferation of the cells; or a different % inhibition, if indicated), for several compounds of the present invention, are determined using this assay.

The results of the above mentioned assays show that several compounds of the invention are able to inhibit the production of VEGF in Hep3B cells at concentrations in the low micromolar range. For example, in the VEGF-Luciferase assay the compound 3-[4-phenylthiazol-2-yl]-7-(N,N-diethylamino)-chromen-2-one of Example 8 shows an IC₅₀ of less than 10 μM.

Specifically claimed compounds as inhibitor of VEGF transcription and production

| | | |
|---|---|---|
| 1 | 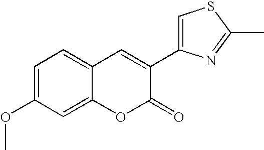 | 7-Methoxy-3-(2-methyl-thiazol-4-yl)-chro- men-2-one |
| 2 | 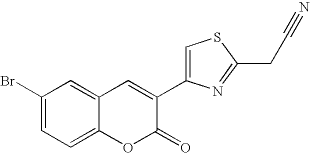 | [4-(6-Bromo-2-oxo-2H-chromen-3-yl)-thia- zol-2-yl]-acetonitrile |
| 3 | 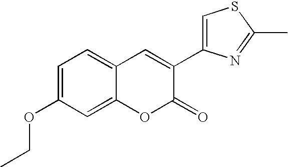 | 7-Ethoxy-3-(2-methyl-thiazol-4-yl)-chro- men-2-one |
| 4 | 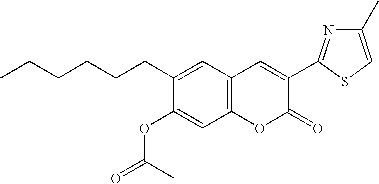 | Acetic acid 6-hexyl-3-(4-methyl-thia- zol-2-yl)-2-oxo-2H-chromen-7-yl ester |
| 5 | 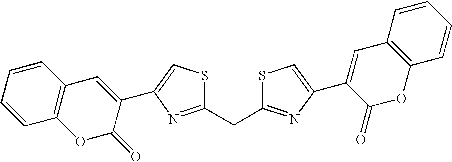 | 3-(2-[4-(Chromen-2-one-3-yl)tiazol-2-yl]thia- zol-4-yl)-chromen-2-one |
| 6 | 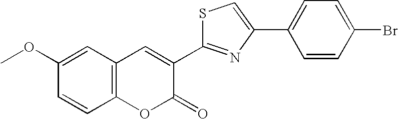 | 7-Methoxy-3-(2-methyl-thiazol-4-yl)-chro- men-2-one |
| 7 | 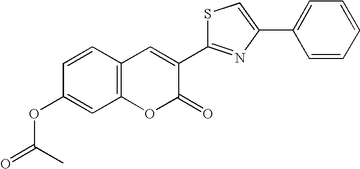 | Acetic acid 2-oxo-3-(4-phenyl-thia- zol-2-yl)-2H-chromen-7-yl ester |
| 8 | 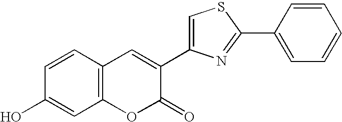 | 7-Hydroxy-3-(2-phenyl-thiazol-4-yl)-chro- men-2-one |
| 9 | 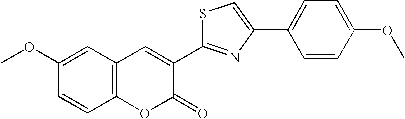 | 6-Methoxy-3-[4-(4-methoxy-phenyl)-thia- zol-2-yl]-chromen-2-one |
| 10 | 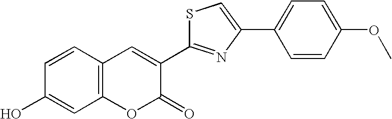 | 7-Hydroxy-3-[4-(4-methoxy-phenyl)-thia- zol-2-yl]-chromen-2-one |
| 11 | 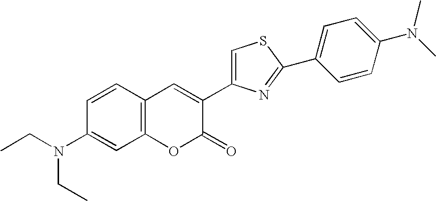 | 7-Diethylamino-3-[2-(4-dimethylamino- phenyl)-thiazol-4-yl]-chromen-2-one |
| 12 | 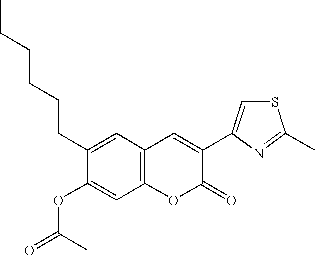 | Acetic acid 6-hexyl-3-(2-methyl- thiazol-4-yl)-2-oxo-2H-chromen-7-yl ester |
| 13 | 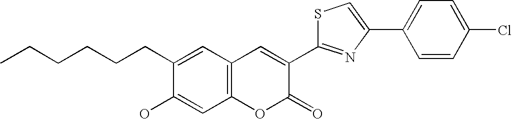 | 3-[4-(4-Chloro-phenyl)-thiazol-2-yl]-6-hex- yl-7-hydroxy-chromen-2-one |
| 14 | 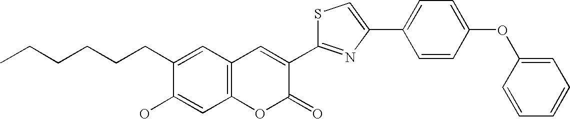 | 6-Hexyl-7-hydroxy-3-[4-(4-phenoxy-phe- nyl)-thiazol-2-yl]-chromen-2-one |
| 15 | 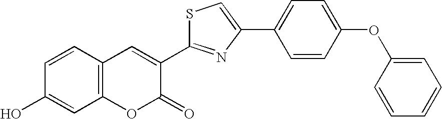 | 7-Hydroxy-3-[4-(4-phenoxy-phenyl)-thia- zol-2-yl]-chromen-2-one |
| 16 | 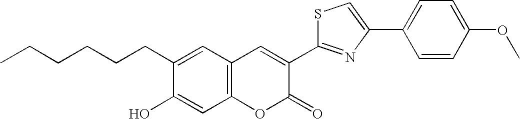 | 6-Hexyl-7-hydroxy-3-[4-(4-methoxy-phe- nyl)-thiazol-2-yl]-chromen-2-one |
| 17 | 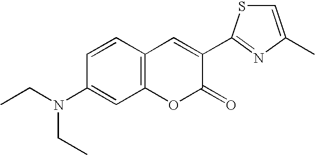 | 7-Diethylamino-3-(4-methyl-thiazol-2-yl)-chro- men-2-one |
| 18 | 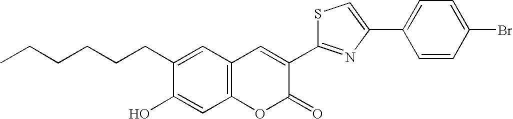 | 3-[4-(4-Bromo-phenyl)-thiazol-2-yl]-6-hex- yl-7-hydroxy-chromen-2-one |
| 19 | 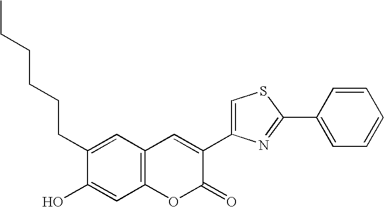 | 6-Hexyl-7-hydroxy-3-(2-phenyl-thia- zol-4-yl)-chromen-2-one |
| 20 | 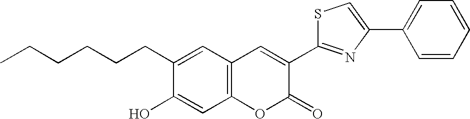 | 6-Hexyl-7-hydroxy-3-(4-phenyl- thiazol-2-yl)-chromen-2-one |
| 21 | 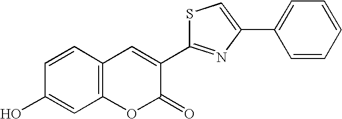 | 7-Hydroxy-3-(4-phenyl-thiazol-2-yl)-chro- men-2-one |
| 22 | 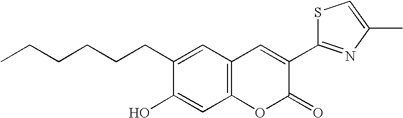 | 6-Hexyl-7-hydroxy-3-(4-methyl- thiazol-2-yl)-chromen-2-one |
| 23 | 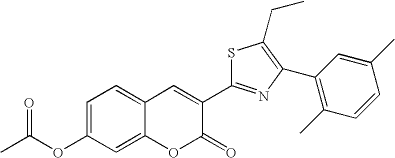 | Acetic acid 3-[4-(2,5-dimethyl-phe- nyl)-5-ethyl-thiazol-2-yl]-2-oxo-2H-chro- men-7-yl ester |
| 24 | 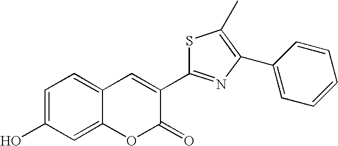 | 7-Hydroxy-3-(5-methyl-4-phenyl- thiazol-2-yl)-chromen-2-one |
| 25 | 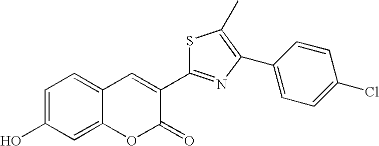 | 3-[4-(4-Chloro-4-phenyl)-5-methyl- thiazol-2-yl]-7-hydroxy-chromen-2-one |
| 26 | 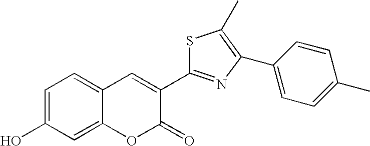 | 7-Hydroxy-3-(5-methyl-4-p-tolyl- thiazol-2-yl)-chromen-2-one |
| 27 | 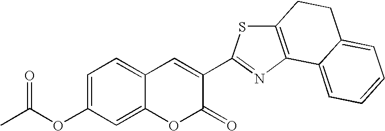 | Acetic acid 3-(4,5-dihydro-naphtho[1,2-d]thia- zol-2-yl)-2-oxo-2H-chromen-7-yl ester |
| 28 | 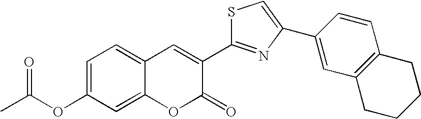 | Acetic acid 2-oxo-3-[4-(5,6,7,8-tetra- hydro-naphthalen-2-yl)-thiazol-2-yl]-2H-chro- men-7-yl ester |
| 29 | 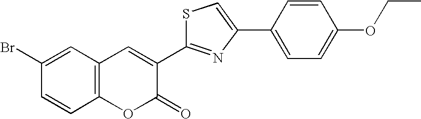 | 6-Bromo-3-[4-(4-ethoxy-phenyl)-thia- zol-2-yl]-chromen-2-one |
| 30 | 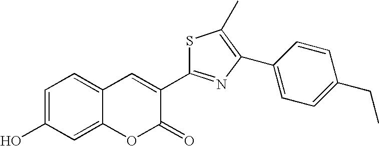 | 3-[4-(4-Ethyl-phenyl)-5-methyl- thiazol-2-yl]-7-hydroxy-chromen-2-one |
| 31 | 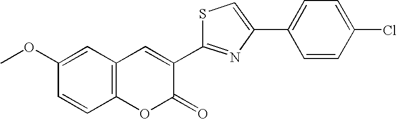 | 3-[4-(4-Chloro-phenyl)-thiazol-2-yl]-6-meth- oxy-chromen-2-one |
| 32 | 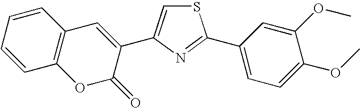 | 3-[2-(3,4-Dimethoxy-phenyl)-thiazol-4-yl]-chro- men-2-one |
| 33 | 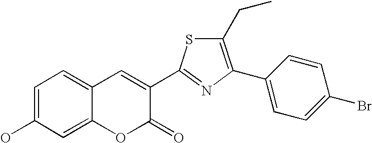 | 3-[4-(4-Bromo-phenyl)-5-ethyl- thiazol-2-yl]-7-hydroxy-chromen-2-one |
| 34 | 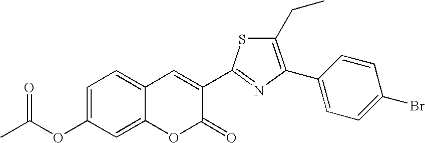 | Acetic acid 3-[4-(4-bromo-phenyl)-5-eth- yl-thiazol-2-yl]-2-oxo-2H-chro- men-7-yl ester |
| 35 | 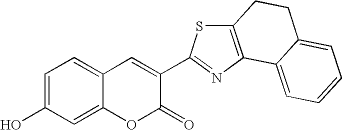 | 3-(4,5-Dihydro-naphtho[1,2-d]thiazol-2-yl)-7-hy- droxy-chromen-2-one |
| 36 | 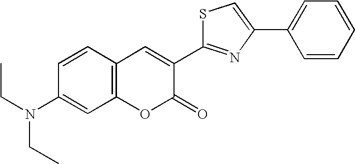 | 7-Diethylamino-3-(4-phenyl-thiazol-2-yl)-chro- men-2-one |
| 37 | 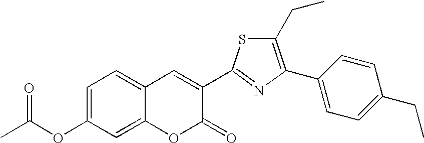 | Acetic acid 3-[5-ethyl-4-(4-ethyl-phe- nyl)-thiazol-2-yl]-2-oxo-2H-chro- men-7-yl ester |
| 38 | 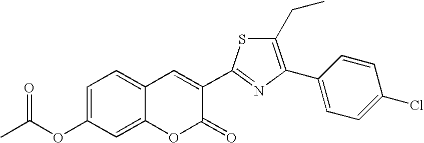 | Acetic acid 3-[4-(4-chloro-phenyl)-5-eth- yl-thiazol-2-yl]-2-oxo-2H-chro- men-7-yl ester |
| 39 | 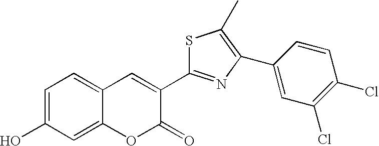 | 3-[4-(3,4-Dichloro-phenyl)-5-methyl- thiazol-2-yl]-7-hydroxy-chromen-2-one |
| 40 | 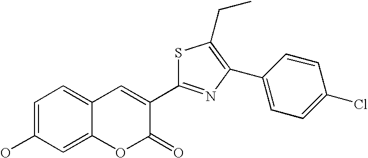 | 3-[4-(4-Chloro-phenyl)-5-ethyl-thia- zol-2-yl]-7-hydroxy-chromen-2-one |
| 41 | 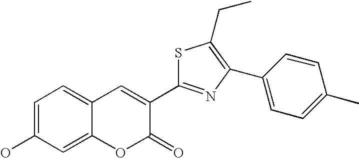 | 3-(5-Ethyl-4-p-tolyl-thiazol-2-yl)-7-hy- droxy-chromen-2-one |
| 42 | 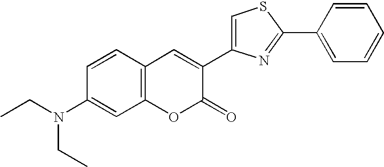 | 7-Diethylamino-3-(2-phenyl-thiazol-4-yl)-chro- men-2-one |
| 43 | 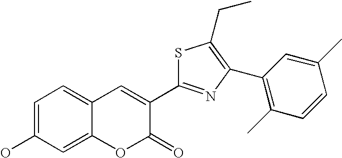 | 3-[4-(2,5-Dimethyl-phenyl)-5-ethyl- thiazol-2-yl]-7-hydroxy-chromen-2-one |
| 44 | 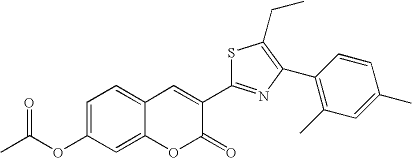 | Acetic acid 3-[4-(2,4-dimethyl- phenyl)-5-ethyl-thiazol-2-yl]-2-oxo-2H-chro- men-7-yl ester |
| 45 | 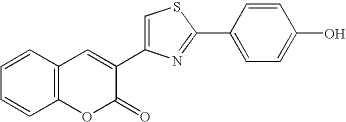 | 3-[2-(4-Hydroxy-phenyl)-thiazol-4-yl]-chro- men-2-one |
| 46 | 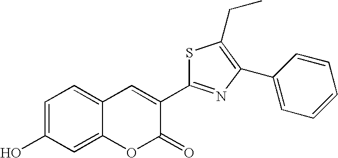 | 3-(5-Ethyl-4-phenyl-thiazol-2-yl)-7-hy- droxy-chromen-2-one |
| 47 | 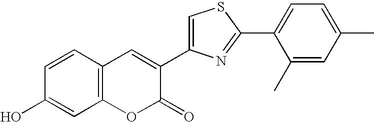 | 3-[2-(2,4-Dimethyl-phenyl)-thiazol-4-yl]-7-hy- droxy-chromen-2-one |
| 48 | 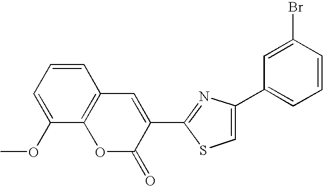 | 3-[4-(3-Bromo-phenyl)-thiazol-2-yl]-8-meth- oxy-chromen-2-one |
| 49 | 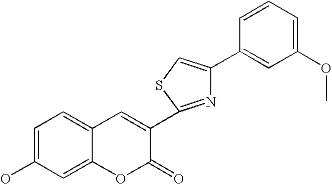 | 7-Hydroxy-3-[4-(3-methoxy-phenyl)-thia- zol-2-yl]-chromen-2-one |
| 50 | 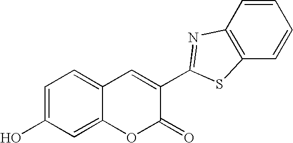 | 3-Benzothiazol-2-yl-7-hydroxy-chro- men-2-one |
| 51 | 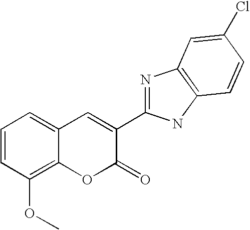 | 3-(5-Chloro-1H-benzoimidazol-2-yl)-8-meth- oxy-chromen-2-one |
| 52 | 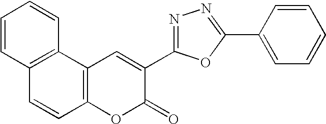 | 2-(5-Phenyl-[1,3,4]oxadiazol-2-yl)-ben- zo[f]chromen-3-one |
| 53 | 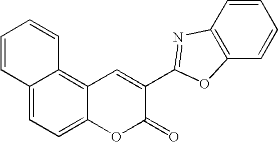 | 2-Benzooxazol-2-yl-benzo[f]chromen-3-one |
| 54 | 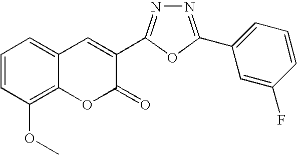 | 3-[5-(3-Fluoro-phenyl)-[1,3,4]oxa- diazol-2-yl]-8-methoxy-chromen-2-one |
| 55 | 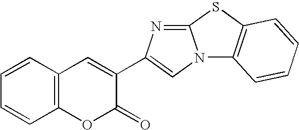 | 3-Benzo[d]imidazo[2,1-b]thiazol-2-yl-chro- men-2-one |
| 56 | 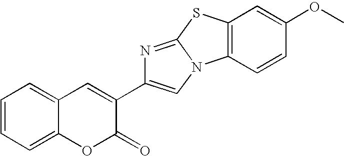 | 3-(7-Methoxy-benzo[d]imidazo[2,1-b]thia- zol-2-yl)-chromen-2-one |
| 57 | 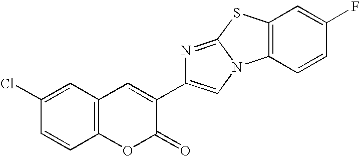 | 6-Chloro-3-(7-fluoro-benzo[d]imi- dazo[2,1-b]thiazol-2-yl)-chromen-2-one |
| 58 | 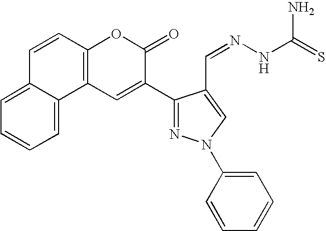 | 2-(4-Thiosemicarbazidomethyl-1-phe- nyl-1H-pyrazol-3-yl)-benzo[f]chromen-3-one |
| 59 | 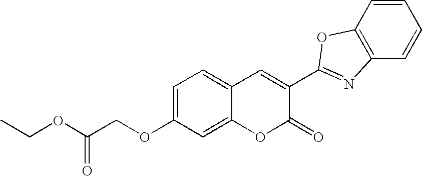 | (3-Benzooxazol-2-yl-2-oxo-2H-chro- men-7-yloxy)-acetic acid ethyl ester |
| 60 | 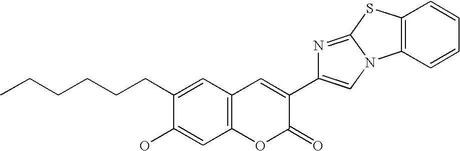 | 3-Benzo[d]imidazo[2,1-b]thiazol-2-yl-6-hex- yl-7-hydroxy-chromen-2-one |
| 61 | 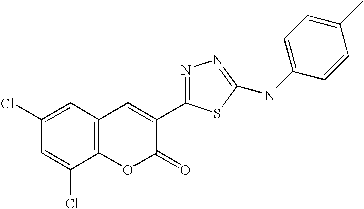 | 6,8-Dichloro-3-(5-p-tolylamino-[1,3,4]thia- diazol-2-yl)-chromen-2-one |
| 62 | 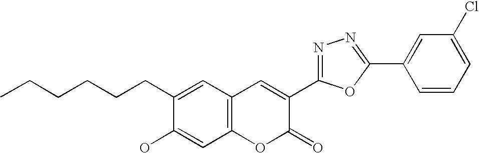 | 3-[5-(3-Chloro-phenyl)-[1,3,4]oxa- diazol-2-yl]-6-hexyl-7-hydroxy-chro- men-2-one |
| 63 | 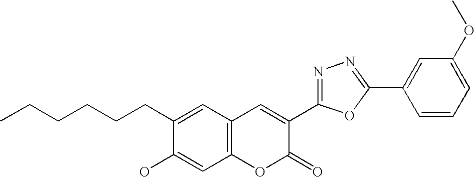 | 6-Hexyl-7-hydroxy-3-[5-(3-methoxy-phe- nyl)-[1,3,4]oxadiazol-2-yl]-chro- men-2-one |
| 64 | 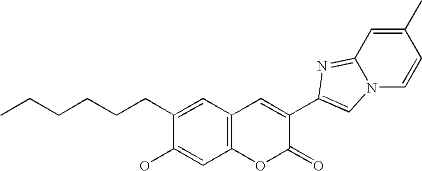 | 6-Hexyl-7-hydroxy-3-(7-methyl- imidazo[1,2-a]pyridin-2-yl)-chromen-2-one |
| 65 | 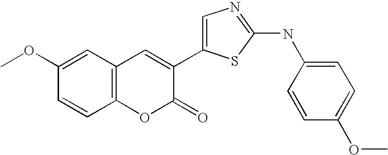 | 6-Methoxy-3-[2-(4-methoxy-phenyl- amino)-thiazol-5-yl]-chromen-2-one |
| 66 | 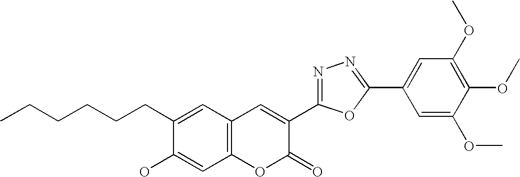 | 6-Hexyl-7-hydroxy-3-[5-(3,4,5-trimeth- oxy-phenyl)-[1,3,4]oxadiazol-2-yl]-chro- men-2-one |

1. Compounds of the formula I 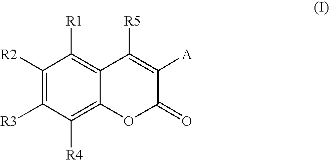
wherein:
A is a four to seven membered heterocyclic ring, aromatic or non aromatic, containing one or more nitrogen, oxygen or sulfur atoms in one or more heterocyclic rings and optionally substituted on the carbon atoms with halogens, alkyls which may be optionally substituted by halogen, amino, hydroxy or cyano groups, aryls, an aromatic or non-aromatic 5- or 6-membered heterocyclic ring containing at least one atom of oxygen, sulfur o nitrogen, hydroxy, amino, monoalkylamino, monoarylamino, bisalkylamino, bisarylamino, (alkyl)(aryl)amino, carbonylamino, alkyl(carbonyl)amino, alkoxycarbonyl, carboxy, cyano groups or, on the nitrogen atoms, with alkyl, aryl, arylalkyl groups or with oxygen atoms to form N-oxides; said four to seven membered heterocyclic ring being optionally fused to one or two aryl, heteroaryl or cycloalkyl groups, in their turn optionally substituted with amino, C1-C8-monoalkylamino, monoarylamino, C1-C8-bisalkylamino, aryloxy, halogens, alkyl, hydroxy, alkoxycarbonyl, carboxy, cyano groups; said aryl, heteroaryl or cycloalkyl groups being optionally partially saturated or unsaturated, respectively;
R1-R4 are independently selected from:
hydrogen, C₁-C₂₀ alkyl optionally interrupted by one or more heteroatoms such as oxygen, sulfur and nitrogen, hydroxy, C₁-C₈ alkoxy, C₁-C₈ alkoxy optionally substituted with hydroxyl, amino, thio, cyano, carboxy, carboxylic esters, or amides, C₁-C₈ haloalkoxy, phenoxy, aralkoxy, C₁-C₈ acyloxy, amino, C₁-C₈ monoalkylamino, C₁-C₈-bisalkylamino, C₁-C₈-acylamino, C₁-C₈-alkylsulfonylamino, aroylamino, halogen, nitro, cyano, trifluoromethyl, carboxy, C₁-C₃ alkoxycarbonyl, a RₐR_{b}N(CH₂)ₙC(═O)— group where Rₐ and R_{b} are independently hydrogen, C₁-C₃-alkyl or Rₐ and R_{b} together with the nitrogen atom they are linked to form a pyrrolidino, piperidino, piperazino or morpholino ring and n=0 or an integer 2 to 4, sulfonyl, mercapto, C₁-C₄-alkylthio, C₁-C₄-alkylsulfonyl, C₁-C₄-alkylsulfinyl, aminosulfonyl, C₁-C₃-alkylaminosulfonyl, a group CH₂NRₐR_{b}, or, taken together with the atoms to which they are attached, R1 and R2 or R2 and R3, or R3 and R4 form an additional aromatic or heteroaromatic ring;
R5 is hydrogen, C₁-C₄-alkyl, C₇-C₁₀ aralkyl,
or a pharmaceutically acceptable salt, solvate, amide, ester, N-oxide, chemically protected form, and prodrug thereof,
as inhibitors of VEGF transcription in mammalian cells.

2. Compounds according to claim 1 wherein the heterocyclic rings A are selected from pyrrolyl, furanyl, thiophenyl, pyrazolyl, thiazolyl, indolyl, oxazolyl, imidazolyl, isothiazolyl, isoxazolyl, 1,2,3-triazolyl, 1,2,4-triazolyl, 1,2,4-oxadiazolyl, 1,3,4-oxadiazolyl, 1,2,5-oxadiazolyl, 1,2,5-thiadiazolyl, 1,3,4-thiadiazolyl, tetrazolyl, pyrimidinyl, pyridazinyle, pyrazinyl, 1,2,4-triazinyl, benzofuranyl, indazolyl, carbazolyl, benzoxazolyl, benzimidazolyl, benzothiazolyl, benzotriazolyl, quinolinyl, isoquinolinyl, cinnolinyl, quinoxalinyl, quinazolinyl, phthalazinyl, 1,2,3-triazinyl, 1,2,4-triazinyl, 1,3,5-triazinyl, purinyl, pteridinyl, benzo[d]imidazo[2,1-b]thiazolyl, 4,5-dihydro-naphtho[1,2-d]thiazolyl, imidazo[1,2-a]pyridinyl.

3. Compounds according to claim 2 wherein A is selected from: thiazolyl, 1,3,4-oxadiazolyl, 1,3,4-thiadiazolyl, benzothiazolyl, benzimidazolyl, benzoxazolyl, benzo[d]imidazo[2,1-b]thiazolyl, 4,5-dihydro-naphtho[1,2-d]thiazolyl, imidazo[1,2-a]pyridinyl.

4. Compounds according to claim 3 wherein A is selected from thiazolyl, wherein the thiazole ring is connected to the 3-position of the coumarin ring through the 2-, 4- or 5-position, i.e. a 2-thiazolyl, 4-thiazolyl or 5-thiazolyl residue, 1,3,4-oxadiazol-2-yl, 1,3,4-thiadiazol-2-yl, benzothiazol-2-yl, benzimidazol-2-yl, benzoxazol-2-yl, benzo[d]imidazo[2,1-b]thiazol-2-yl of formula 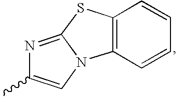
4,5-dihydro-naphtho[1,2-d]thiazole-2-yl of formula 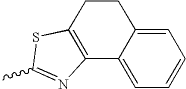
imidazo[1,2-a]pyridine-2-yl of formula

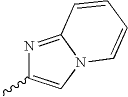

5. Compounds according to any one of claims 1 to 4 wherein R1, R2, R3, and R4 are hydroxy, C1-C8-alkoxy, amino, C₁-C₈ monoalkylamino, C₁-C₈-bisalkylamino.

6. Compounds according to claim 5, wherein R1, R2, R3, and R4 are hydroxy or diethylamino.

7. A compound according to claim 1, which is 3-[4-phenylthiazol-2-yl]-7-(N,N-diethylamino)-chromen-2-one.

8. Compounds according to claims 1-7 as angiogenesis inhibitors.

9. Compounds according to claims 1-7 as anti-proliferative agents.

10. A composition comprising a compound as defined in claims 1-7 and a pharmaceutically acceptable carrier.

11. A method of inhibiting VEGF production in a cell, comprising contacting said cell with an effective amount of an active compound, as defined in claims 1-7.

12. A method of inhibiting angiogenesis, comprising contacting a cell with an effective amount of an active compound, as defined in claims 1-7, whether in vitro or in vivo.

13. A method of treating a proliferative condition in a patient comprising administering to said patient a therapeutically-effective amount of an active compound, as defined in claims 1-7.

14. The use of an active compound, as defined in claims 1-7 for the manufacture of a medicament for use in the treatment of a proliferative condition.
